(19) United States
(12) Patent Application Publication
(10) Pub. No.: US 2021/0259539 A1
(43) Pub. Date: Aug. 26, 2021

(54) SYSTEMS, METHODS, AND COMPUTER PROGRAM PRODUCTS FOR VISION ASSESSMENTS USING A VIRTUAL REALITY PLATFORM

(71) Applicant: Allergan, Inc., Irvine, CA (US)

(72) Inventors: Amber Lewis, Newport Beach, CA (US); Francisco J. Lopez, Ladera Ranch, CA (US); Gaurang Patel, Irvine, CA (US)

(21) Appl. No.: 17/180,130

(22) Filed: Feb. 19, 2021

Related U.S. Application Data
(60) Provisional application No. 62/979,575, filed on Feb. 21, 2020.

(51) Int. Cl. A61B 3/032; A61B 3/00; G06F 3/01; G06F 3/16; G06T 19/00; G02B 27/00; G02B 27/01

(52) U.S. Cl. CPC A61B 3/032; A61B 3/0058; G06F 3/013; G02B 27/0172; G06T 19/003; G06T 19/006; G02B 27/0093; G06F 3/167

(57) ABSTRACT

Methods and Systems for evaluating visual impairment of a user. The methods and systems including generating, using a processor, a virtual reality environment; displaying at least portions of the reality environment on a head-mounted display, and measuring the performance of a user as user interacts with the virtual reality environment using at least one performance metric. Non-transitory computer readable storage medium comprising a sequence of instructions for a processor to execute the methods discussed herein.

### CROSS-REFERENCE TO RELATED APPLICATIONS

This application claims the benefit under 35 U.S.C. § 119(e) of U.S. Provisional Patent Application No. 62/979,575, filed Feb. 21, 2020, and titled “SYSTEMS, METHODS, AND COMPUTER PROGRAM PRODUCTS FOR VISION ASSESSMENTS USING A VIRTUAL REALITY PLATFORM,” the entirety of which is incorporated herein by reference.

### FIELD OF THE INVENTION

The invention relates to vision assessments, particularly functional vision assessments using virtual reality.

### BACKGROUND OF THE INVENTION

Assessment of vision in patients with inherited retinal diseases, such as Leber congenital amaurosis (“LCA”), retinitis pigmentosa, or other conditions with very low vision is a significant challenge in the clinical trial setting. LCA is a group of ultra-rare inherited retinal dystrophies characterized by profound vision loss beginning in infancy. LCA10 is a subtype of LCA that accounts for over 20% of all cases and is characterized by mutations in the CEP290 (centrosomal protein 290) gene. Most patients with LCA10 have essentially no rod-based vision but retain a central island of poorly functioning cone photoreceptors. This results in poor peripheral vision, nyctalopia (night blindness), and a wide range of visual acuities ranging from No Light Perception (“NLP”) to approximately 20/50 vision.

Physical navigation courses have been used in, for example, clinical studies to assess functional vision in patients with low vision. For example, the Multi-luminance Mobility Test (“MLMT”) is a physical navigation course designed to assess functional vision at various light levels in patients with a form of LCA caused by a mutation in the RPE65 gene (LCA2). A similar set of four navigation courses (Ora® Mobility Courses) was designed by Ora®, Inc. and used in LCA10 clinical trials. Although physical navigation courses provide a valuable measurement of visual impairment, they require large dedicated spaces, time-consuming illuminance calibration, time and labor to reconfigure the course, and manual (subjective) scoring. Equipment systems and methods are thus desired to conduct functional vision assessments for use in, for example, clinical studies that avoid the disadvantages of these physical navigation courses.

### SUMMARY OF THE INVENTION

One aspect of the present invention has been developed to avoid disadvantages of the physical navigation courses discussed above using a virtual reality environment. Although this aspect of the present invention has various advantages over the physical navigation courses, the invention is not limited to embodiments of functional vision assessment in patients with low vision disorders discussed in the background. As will be apparent from the following disclosure, the devices, systems, and methods discussed herein encompass many aspects of using a virtual reality environment for the assessment of vision in individuals.

In one aspect, the invention relates to a method of evaluating visual impairment of a user including: generating, using a processor, a virtual navigation course for the user to navigate; displaying portions of the virtual navigation course on a head-mounted display as the user navigates the virtual navigation course, the head-mounted display being communicatively coupled to the processor; and measuring the progress of the user as user navigates the virtual navigation course using at least one performance metric.

In another aspect, the invention relates to a method of evaluating visual impairment of a user including: generating, using a processor, a virtual reality environment including a virtual object having a directionality; displaying the virtual reality environment including the virtual object on a head-mounted display, the head-mounted display being communicatively coupled to the processor; increasing, using the processor, the size of the virtual object displayed on the head-mounted display; and measuring at least one performance metric when the processor receives an input that a user has indicated the directionality of the virtual object.

In a further aspect, the invention relates to a method of evaluating visual impairment of a user including generating, using a processor, a virtual reality environment including a virtual eye chart located on a virtual wall. The virtual eye chart has a plurality of lines each of which include at least one alphanumeric character. The at-least-one alphanumeric character in a first line of the eye chart is a different size than the at-least-one alphanumeric character in a second line of the eye chart. The method further includes: displaying the virtual reality environment including the virtual eye chart and virtual wall on a head-mounted display, the head-mounted display being communicatively coupled to the processor; displaying, on a head-mounted display, an indication in the virtual reality environment to instruct a user to read one line of the eye chart; and measuring the progress of the user as user reads the at-least-one alphanumeric character of the line of the eye chart using at least one performance metric.

In still another aspect, the invention relates to a method of evaluating visual impairment of a user including: generating, using a processor, a virtual reality environment including a target; displaying the virtual reality environment including the target on a head-mounted display, the head-mounted display being communicatively coupled to the processor and including eye-tracking sensors; tracking the center of the pupil with the eye-tracking sensors to generate eye tracking data as the user stares at the target; and measuring the visual impairment of the user based on the eye tracking data.

In yet another aspect, the invention relates to a method of evaluating visual impairment of a user including: generating, using a processor, a virtual reality environment including a virtual scene having a plurality of virtual objects arranged therein; displaying the virtual reality environment including the virtual scene and the plurality of virtual objects on a head-mounted display, the head-mounted display being communicatively coupled to the processor; and measuring the performance of the user using at least one performance metric when the processor receives an input that a user has selected an object of the plurality of virtual objects.

In still a further aspect, the invention relates to a method of evaluating visual impairment of a user including: generating, using a processor, a virtual driving course for the user to navigate; displaying portions of the virtual driving course on a head-mounted display as the user navigates the virtual navigation course, the head-mounted display being communicatively coupled to the processor; and measuring the progress of the user as user navigates the virtual navigation course using at least one performance metric.

Additional aspects of these inventions also include non-transitory computer readable storage media having stored thereon sequences of instruction for a processor to execute the forgoing methods and those discussed further below. Similarly, additional aspects of the invention include systems configured to be used in conjunction with these methods.

These and other aspects of the invention will become apparent from the following disclosure.

### BRIEF DESCRIPTION OF THE DRAWINGS

FIG. 1 is a schematic block diagram of a virtual reality system according to a preferred embodiment of the invention.

FIG. 2 shows a head-mounted display of the virtual reality system on the head of a user.

FIG. 3 shows a left controller of a pair of controllers of the virtual reality system in the left hand of a user.

FIG. 4 is a schematic of a user in a physical room in which the user uses a virtual reality system according to a preferred embodiment of the invention.

FIG. 5 shows an underside of the head-mounted display of the virtual reality system on the head of a user.

FIG. 6 shows a nose insert for the head-mounted display.

FIG. 7 shows the nose insert shown in FIG. 6 installed in the head-mounted display.

FIG. 8 is a perspective view of a first virtual room of a virtual navigation course according to a preferred embodiment of the invention.

FIG. 9 is a plan view taken from above of the first virtual room shown in FIG. 8.

FIG. 10 shows an integrated display of the head-mounted display with the user in a first position in the first virtual room shown in FIG. 8.

FIG. 11 shows an integrated display of the head-mounted display with the user in a second position in the first virtual room shown in FIG. 8.

FIG. 12 shows an integrated display of the head-mounted display with the user in a third position in the first virtual room shown in FIG. 8.

FIG. 13 shows an integrated display of the head-mounted display with the user in a fourth position in the first virtual room shown in FIG. 8.

FIG. 14 is a perspective view of a second virtual room of the virtual navigation course according to a preferred embodiment of the invention.

FIG. 15 is a plan view taken from above of the second virtual room shown in FIG. 14.

FIG. 16 shows an integrated display of the head-mounted display with the user in a first position in the second virtual room shown in FIG. 14.

FIG. 17 shows an integrated display of the head-mounted display with the user in a second position in the second virtual room shown in FIG. 14.

FIG. 18 shows an integrated display of the head-mounted display with the user in a third position in the second virtual room shown in FIG. 14.

FIG. 19 shows an integrated display of the head-mounted display with the user in a fourth position in the second virtual room shown in FIG. 14.

FIG. 20 shows an integrated display of the head-mounted display with the user in a fifth position in the second virtual room shown in FIG. 14.

FIG. 21 shows an integrated display of the head-mounted display with the user in a sixth position in the second virtual room shown in FIG. 14.

FIG. 22 is a perspective view of a third virtual room of the virtual navigation course according to a preferred embodiment of the invention.

FIG. 23 is a plan view taken from above of the third virtual room shown in FIG. 22.

FIG. 24 shows an integrated display of the head-mounted-display with the user in a first position in the third virtual room shown in FIG. 22.

FIG. 25 shows an integrated display of the head-mounted display with the user in a second position in the third virtual room shown in FIG. 22.

FIG. 26 shows an integrated display of the head-mounted display with the user in a third position in the third virtual room shown in FIG. 22.

FIG. 27 shows an integrated display of the head-mounted display with the user in a fourth position in the third virtual room shown in FIG. 22.

FIG. 28 shows an integrated display of the head-mounted display with the user in a fifth position in the third virtual room shown in FIG. 22.

FIG. 29 shows an integrated display of the head-mounted display with the user in a sixth position in the third virtual room shown in FIG. 22.

FIG. 30 illustrates simulated impairment conditions used in a study using the virtual navigation course.

FIG. 31 are LSmeans±SE derived from a mixed model repeated measures analysis for time to complete the virtual navigation course.

FIG. 32 are LSmeans±SE derived from a mixed model repeated measures analysis for total distance traveled to complete the virtual navigation course.

FIG. 33 are LSmeans±SE derived from a mixed model repeated measures analysis for number of collisions with virtual objects when completing the virtual navigation course.

FIG. 34 are scatter plots of results of the study comparing an initial test to a retest as well as linear regression with the shaded area representing the 95% confidence bounds for the time to complete the virtual navigation course.

FIG. 35 are Bland-Altman plots of results of the study for the time to complete the virtual navigation course.

FIG. 36 are scatter plots of results of the study comparing an initial test to a retest as well as linear regression with the shaded area representing the 95% confidence bounds for the total distance traveled to complete the virtual navigation course.

FIG. 37 are Bland-Altman plots of results of the study for the total distance traveled to complete the virtual navigation course.

FIG. 38 are scatter plots of results of the study comparing an initial test to a retest as well as linear regression with the shaded area representing the 95% confidence bounds for the number of collisions with virtual objects when completing the virtual navigation course.

FIG. 39 are Bland-Altman plots of results of the study for the number of collisions with virtual objects when completing the virtual navigation course.

FIGS. 40A-40C illustrate the virtual reality environment for a first task in a low-vision visual acuity assessment according to another preferred embodiment of the invention. FIG. 40A is an initial size of an alphanumeric character used in the first task of the virtual reality environment of this embodiment. FIG. 40B is a second size (a medium size) of the alphanumeric character used in the first task of the virtual reality environment of this embodiment. FIG. 40C is a third size (a largest size) of the alphanumeric character used in the first task of the virtual reality environment of this embodiment.

FIG. 41 shows an alphanumeric character that may be used in the low vision visual acuity assessment.

FIG. 42 shows another alphanumeric character that may be used in the low vision visual acuity assessment.

FIGS. 43A-43C illustrate the virtual reality environment a second task in the low vision visual acuity assessment. FIG. 43A is an initial width of initial width of bars of the grating used in the second task of the virtual reality environment of this embodiment. FIG. 43B is a second width of bars of the grating used in the second task of the virtual reality environment of this embodiment. FIG. 43C is a third width of bars of the grating used in the second task of the virtual reality environment of this embodiment.

FIG. 44 illustrates the virtual reality environment of a visual acuity assessment in a further preferred embodiment of the invention.

FIGS. 45A-45C illustrate alternate targets in a virtual reality environment of the oculomotor instability assessment.

FIGS. 46A and 46B show an example virtual reality scenario used in an item search assessment according to still another preferred embodiment of the invention. FIG. 46A is a high (well-lit) luminance level, and FIG. 46B is a low (poorly lit) luminance level.

FIGS. 47A and 47B show another example virtual reality scenario used in the item search assessment. FIG. 47A is a high (well-lit) luminance level, and FIG. 47B is a low (poorly lit) luminance level.

FIG. 48 shows a further example virtual reality scenario used in the item search assessment.

FIG. 49 shows a still another example virtual reality scenario used in the item search assessment.

FIGS. 50A and 50B show an example virtual reality environment used in a driving assessment according to yet another preferred embodiment of the invention. FIG. 50A is a high (well-lit) luminance level, and FIG. 50B is a low (poorly lit) luminance level.

FIGS. 51A and 51B show another example virtual reality environment used in a driving assessment. FIG. 51A is a high (well-lit) luminance level, and FIG. 51B is a low (poorly lit) luminance level.

### DETAILED DESCRIPTION OF THE PREFERRED EMBODIMENTS

In a preferred embodiment of the invention, a functional vision assessment is conducted using a virtual reality system 100 and a virtual reality environment 200 developed for this assessment. In one embodiment, the functional vision assessment is a navigation assessment using a virtual navigation course 202. The virtual navigation course 202 may be used to assess the progression of a patient's disease or the efficacy or benefit of his or her treatment. The patient or user 10 navigates the virtual navigation course 202, and the time to completion and various other performance metrics can be measured to determine the patient's level of visual impairment; those metrics can also be stored and compared across repeated navigations by the patient (user 10).

A virtual navigation course 202 has technical advantages over physical navigation courses. For example, the virtual reality navigation course 202 of this embodiment is readily portable. The virtual navigation course 202 only requires a virtual reality system 100 (including for example a head-mounted display 110 and controllers 120) and a physical room 20 of sufficient size to use the virtual reality system 100. In contrast, the physical navigation course requires all the components and objects in the room to be shipped to and stored onsite. The physical room 20 used for the virtual reality navigation course can be a smaller size than the room used for the physical navigation courses. “Installation” or setup of the virtual navigation course 202 is as simple as starting up the virtual reality system 100 and offers the ability for instant, randomized course reconfiguration. In contrast, the physical navigation courses are time- and labor-intensive to install and reconfigure. Additionally, the environment the patient sees in the virtual navigation course can be adjusted in numerous ways that can be used in the visual impairment evaluation, including by varying the illumination and brightness levels, as discussed below, the chromatic range, and other controlled image patterns that would be difficult to precisely change and measure in a non-virtual environment.

Another disadvantage of the physical navigation courses is a time-consuming process to calibrate the illuminance of the course correctly. When the physical navigation course is established, a lighting calibration is conducted at about one-foot increments along the total length of the path of the physical maze. This calibration this then repeated in this one-foot increment for every different level of light for which the physical navigation course will be used. In addition, spot verification needs to be performed periodically (such as each day of testing) to confirm that the physical navigation course is property calibrated and the conditions have not changed. In contrast, the virtual reality environment 200 and virtual reality system 100 offer complete control of lighting conditions without the need for frequent recalibration. The head-mounted display 110 physically prevents light leakage from the surrounding environment ensuring consistency across clinical trial sites. Luminance levels of varying difficulty are determined mathematically by the virtual reality system 100. The luminance levels can be verified empirically using, for example, a spot photometer (such as ColorCal MKII Colorimeter by Cambridge Research Systems Ltd. of Kent, United Kingdom). This empirical verification can be performed by placing the spot photometer over the integrated display 112 of the head-mounted display 110 while the virtual reality system 100 systematically renders different lighting conditions within the exact same virtual scene.

Moreover, scoring for the physical navigation course is done by physical observation by two independent graders and thus is a subjective scoring system with inherent uncertainty. In embodiments discussed herein, the scoring is assessed by the virtual reality system 100 and thus provides more objective scoring, resulting in a more precise assessment of a patient's performance and the progress of his or her disease or treatment. A further cumulative benefit of these advantages is a shorter visit for the patient. In the virtual reality system 100, virtual navigation courses 202 can be customized for each patient without the need for physical changes to the room. Moreover, the system may also be used for visual impairment therapy, whereby the course configurations can be gradually changed as the patient makes progress on improving his or visual impairment. These and other advantages of this preferred embodiment of the invention will become apparent from the following disclosure.

Still a further advantage of the virtual navigation course 202 over a physical navigation course is that the virtual navigation course 202 can be readily used by patients (users 10) that have physical disabilities other than their vision. For example, a user 10 that is in a wheelchair or a walking assist device (e.g., walker or crutches) can easily use the virtual navigation course 202, but the typical physical navigation course does not allow for such patients.

### Virtual Reality System

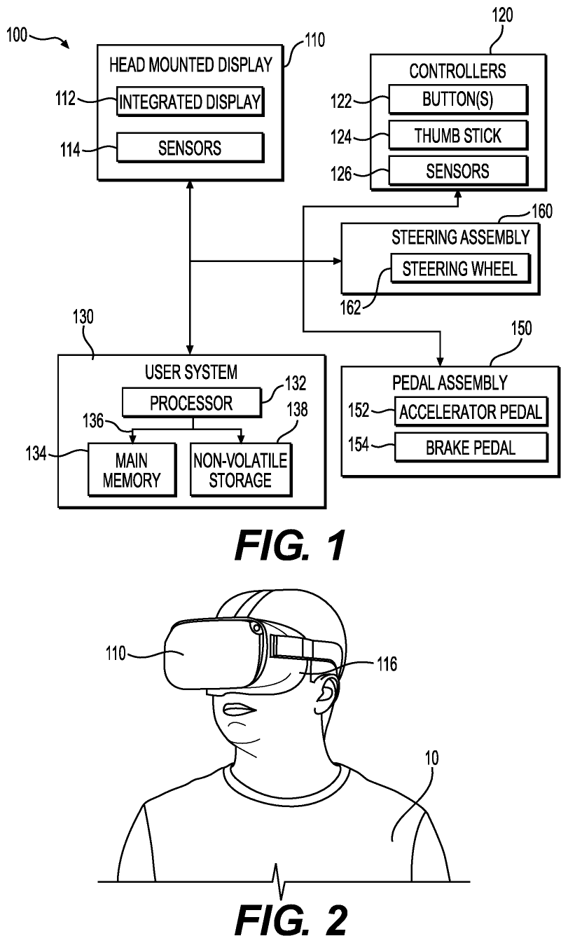

The vision assessments discussed herein are performed using a virtual reality system 100. Any suitable virtual reality system 100 may be used. For example, Oculus® virtual reality systems, such as the Oculus Quest®, or the Oculus Rift® made by Facebook Technologies of Menlo Park, Calif., may be used. In another example, the HTC Vive® virtual reality systems, including the HTC Vive Focus®, HTC Vive Focus Plus®, HTC Vive Pro Eye®, and HTC Vive Cosmos® headsets, made by HTC Corporation of New Taipei City, Taiwan, may be used. Other virtual reality systems and head-mounted displays, such as Windows Mixed Reality systems, may also be used. FIG. 1 is a schematic block diagram of the virtual reality system 100 of this embodiment. The virtual reality system 100 includes a head-mounted display 110, a pair of controllers 120 and a user system 130.

The head-mounted display 110 and the user system 130 are described herein as separate components, but the virtual reality system 100 is not so limited. For example, the head-mounted display 110 may incorporate some or all of the functionality associated with the user system 130. In addition, various functionality and components that are shown in this embodiment as part of the head-mounted display 110, the controller 120, and the user system 130 may be separate from these components. For example, sensors 114 are described as being part of the head-mounted display 110 to track and determine the position and movement of the user 10 and, in particular, the head of the user 10, the hands of the user 10, and/or controllers 120. Such tracking is sometimes referred to as inside-out tracking. However, some or all of the functionality of the sensors 114 may be implemented by sensors located on the physical walls 22 of a physical room 20 (see FIG. 4) in which the user 10 uses the virtual reality system 100. Other sensor configurations are possible, such as by using a front facing camera or eye-level placed sensors.

FIG. 2 shows the head-mounted display 110 on the head of a user 10. The head-mounted display 110 may also be referred to as a virtual reality (VR) headset. As can be seen in FIG. 2, the user 10 is a person who is wearing the head-mounted display 110. The head-mounted display 110 includes an integrated display 112 (see FIG. 1), and the user 10 wears the head-mounted display 110 in such a way that he or she can see the integrated display 112. In this embodiment, the head-mounted display 110 is positioned on the head of the user 10 with integrated display 112 positioned in front of the eyes of the user 10. Also in this embodiment, the integrated display 112 has two separate displays, one for each eye. However, the integrated display 112 is not so limited and any number of displays may be used. For example, a single display may be used as the integrated display 112, such as when the display of a mobile phone is used.

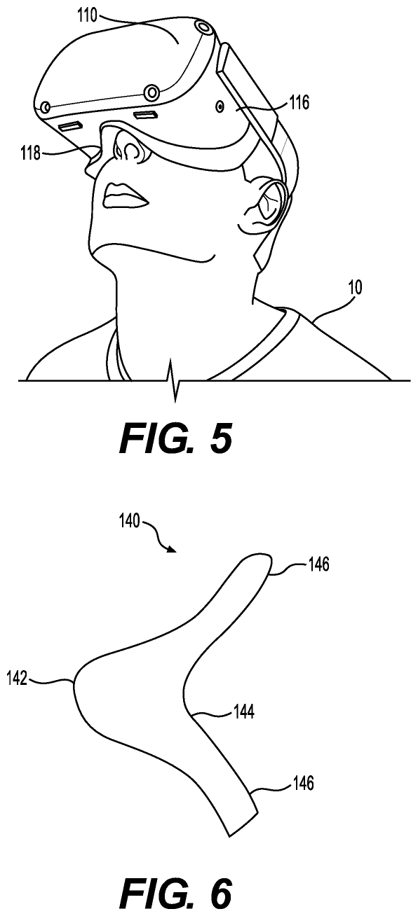

In this embodiment, the head-mounted display 110 includes a facial interface 116. The facial interface 116 is a facial interface foam that surrounds the eyes of the user 10 and prevents at least some of the ambient light from the physical room 20 from entering a space between the eyes of the user 10 and the integrated display 112. The facial interface 116 of many of the commercial head-mounted displays 110, such as those discussed above, are contoured to fit the face of the user 10 and fit over the nose of the user 10. In some cases, the facial interface 116 is contoured to have a nose hole such that a gap 118 is formed between the nose of the user 10 and the facial interface 116, as can be seen in FIG. 5. (Reference numeral 118 will be used to refer to both the nose hole and gap herein.) As discussed herein, the virtual reality environment 200 is carefully calibrated for various lighting conditions. The presence of the gap 118 may allow ambient light to enter the head-mounted display 110 and alter the lighting conditions. To avoid this, a nose insert 140 may be used to block the ambient light.

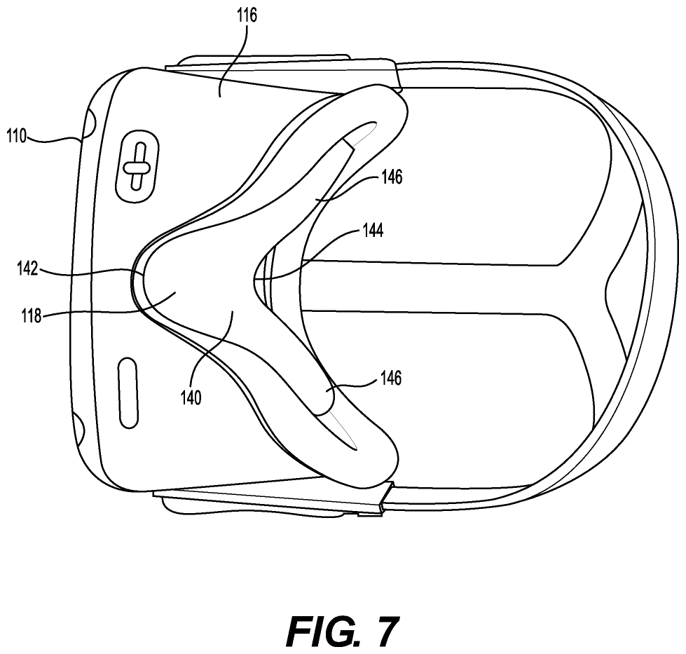

The nose insert 140 is shown in FIG. 6 and an underside of the head-mounted display 110 with the nose insert 140 installed is shown in FIG. 7. The nose insert 140 of this embodiment is a compressible piece of foam that is cut to fit in the nose hole 118 of the facial interface 116. As can be seen in FIG. 6, the nose insert 140 has a convex surface 142, which in this embodiment has a parabolic shape. The convex surface 142 of the nose insert 140 is sized to fit snuggly within the nose hole 118 and shaped to fit the contour of the facial interface 116. The nose insert 140 also includes a concave surface 144 on the opposite side of the convex surface 142. The concave surface 144 also has a parabolic shape in this embodiment and will be the portion of the nose insert 140 that is in contact with the nose of the user 10. To help hold the nose insert 140 in place and fill any gaps between the facial interface 116 and the cheeks of the user 10, the nose insert 140 also includes a pair of flanges 146 on either side of the concave surface 144. As discussed above, the nose insert 140 of this embodiment is compressible such that, when the head-mounted display 110 is on the face of the user 10, the nose insert 140 is compressed between the face (nose and cheeks) of the user 10 and the facial interface 116, blocking ambient light from entering.

As shown in FIG. 1 and noted above, the head-mounted display 110 of this embodiment also includes one or more sensors 114 that may be used to generate motion, position, and orientation data (information) for the head-mounted display 110 and the user 10. Any suitable motion, position, and orientation sensors may be used, including, for example, gyroscopes, accelerometers, magnetometers, video cameras, and color sensors. These sensors 114 may include, for example, those used with “inside-out tracking” where sensors within the headset, including cameras, are used to track the user's movement and position within the virtual environment. Other tracking solutions can involve a series of markers, such as reflectors, lights, or other fiducial markers, are placed on the physical walls 22 of the physical room 20. When viewed by a camera or other sensors mounted on the head-mounted display 110, these markers provide one or more points of reference for interpolation by software in order to generate motion, position, and orientation data.

In this embodiment, the sensors 114 are located on the head-mounted display 110, but location of the sensors 114 is not so limited and the sensors 114 may be placed in other locations. FIG. 4 shows the user 10 in a physical room 20 in which the user 10 uses the virtual reality system 100. The virtual reality system 100 shown in FIG. 4 includes sensors 114 mounted on the physical walls 22 of the physical room 20 that are used to determine the motion, position, and orientation of the head-mounted display 110 and the user 10. Such external sensors 114 may include, for example, a camera or color sensor that detects a series of markers, such as reflectors or lights (e.g., infrared or visible light), that, when viewed by an external camera or illuminated by a light, may provide one or more points of reference for interpolation by software in order to generate motion, position, and orientation data.

As show schematically in FIG. 1, the user system 130 is a computing device that is used to generate a virtual reality environment 200 (discussed further below) for display on the head-mounted display 110 and, in the embodiments discussed herein, the virtual navigation course 202. The user system 130 of this embodiment includes a processor 132 connected to a main memory 134 through, for example, a bus 136. The main memory 134 stores, among other things, instructions and/or data for execution by the processor 132. The main memory 134 may include read-only memory (ROM) or random access memory (RAM), as well as cache memory. The processor 132 can include any general-purpose processor and a hardware module or software module configured to control the processor 132. The processor 132 may also be a special-purpose processor where software instructions are incorporated into the actual processor design. The processor 132 may be a self-contained computing system, containing multiple cores or processors, a bus, memory controller, cache, etc. A multi-core processor may be symmetric or asymmetric. The user system 130 may also be implemented with more than one processor 132 or on a group or cluster of computing devices networked together to provide greater processing capability.

The user system 130 also includes non-volatile storage 138 connected to the processor 132 and main memory 134 through the bus 136. The non-volatile storage 138 provides non-volatile storage of computer-readable instructions, data structures, program modules, and other data for the user system 130. These instructions, data structures, and program modules include those used in generating the virtual reality environment 200, which will be discussed below, and those used to carry out the vision assessments, also discussed further below. Typically, the data, instructions, and program modules stored in the non-volatile storage 138 are loaded into the main memory 134 for execution by the processor 132. The non-volatile storage 138 may be any suitable non-volatile storage including, for example, solid state memory, magnetic memory, optical memory, and flash memory.

When the user system 130 is co-located with the head-mounted display 110, the integrated display 112 may be directly connected to the processor 132 by the bus 136. Alternatively, the user system 130 may be commutatively coupled to the head-mounted display 110, including the integrated display 112, using any suitable interface. For example, either wired or wireless connections to the user system 130 may be possible. Suitable wired communication interfaces include USB®, HDMI, DVI, VGA, fiber optics, DisplayPort®, Lightening connectors, and ethernet, for example. Suitable wireless communication interfaces include, for example, Wi-Fi®, a Bluetooth®, and radio frequency communication. The head-mounted display 110 and user system 130 shown in FIG. 4 are an example of a tethered virtual reality system 100 where the virtual reality system 100 is connected by a wired interface to a computer operating as the user system 130. Examples of user system 130 include a typical desktop computer (as shown in FIG. 4), a tablet, mobile phone, and a game console, such as the Microsoft® Xbox® and the Sony® PlayStation®.

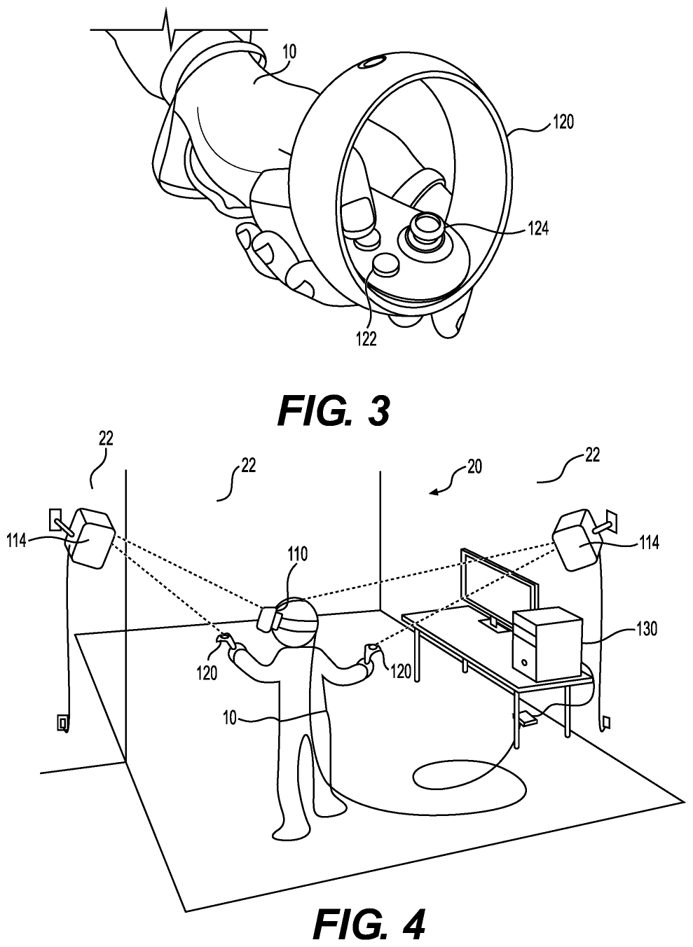

The user system 130 may determine the position, orientation, and movement of the user 10 based on the sensors 114 for the head-mounted display 110 alone, and subsequently adjust what is displayed on the integrated display 112 based on this determination. The user system 130 and processor 132 communicatively coupled to the sensors 114 and configured to receive data from the sensors 114. The virtual reality system 100 of this embodiment, however, also optionally includes a pair of controllers 120. FIG. 3 shows a left controller of the pair of controllers 120 in the hand of a user 10 (see also FIG. 4). The pair of controllers 120 in this embodiment are symmetrical and designed to be used in the left and right hands of the user 10. The virtual reality system 100 can also be implemented without controllers 120 or a single controller 120. The following discussion will refer to the controller 120 and may refer to either one or both controllers of the pair of controllers 120. The controller 120 is communicatively coupled to the user system 130 and the processor 132 using any suitable interface, including, for example, the wired or wireless interfaces discussed above in reference to the connection between the head-mounted display 110 and the user system 130.

The controller 120 of this embodiment includes various features to enable a user to interface with the virtual reality system 100 and virtual reality environment 200. These user interfaces may include a button 122 such as the “X” and “Y” button shown in FIG. 3, which may be selected by the thumb of the user 10, or a trigger button (not shown) on the underside of the body of the controller that may be operated by the index finger of the user 10. Another example of a user interface is a thumb stick 124. As shown schematically in FIG. 1, the controller 120 may also include sensors 126 that can be used by the processor 132 to determine the position, orientation, and movement of the hands of the user 10. Any suitable sensor may be used, including those discussed above, as suitable sensors 114 for the head-mounted display 110. Also, as with the sensors 114 for the head-mounted display 110, the sensors 126 for the controller 120 may be externally located such as on the physical walls 22 of the physical room 20. The controller 120 is communicatively coupled to the user system 130 including the processor 132, and thus the processor 132 is configured to receive data from the sensors 126 and user input from the user interfaces including the button 122 and thumb stick 124.

In some embodiments discussed herein, the user 10 walks through a physical room 20 as they navigate a virtual room 220 (discussed further below). However, the invention is not so limited and user 10 may navigate the virtual room 220 using other methods. In one example, the user 10 may be stationary (either standing or sitting) and navigate the virtual room 220 by using the thumb stick 124 or other controls of the controller 120. In another example, the user 10 may move through the virtual room 220 as they walk on a treadmill.

In one aspect, hardware that performs a particular function includes a software component (e.g., computer-readable instructions, data structures, and program modules) stored in a non-volatile storage 138 in connection with the necessary hardware components, such as the processor 132, main memory 134, bus 136, integrated display 112, sensors 114 for the head-mounted display 110, button 122, thumb stick 124, sensors 126 for the controller 120, and so forth, to carry out the function. In another aspect, the system can use a processor and computer-readable storage medium to store instructions which, when executed by the processor, cause the processor to perform a method or other specific actions. The basic components and appropriate variations are contemplated depending on the type of device, such as whether the user system 130 is implemented on a small, hand-held computing device, a standalone headset, or on a desktop computer, or a computer server.

### Virtual Reality Navigation Course

In a preferred embodiment of the invention, the functional vision assessment is performed using a navigation course developed in a virtual reality environment 200, which may be referred to herein as a virtual navigation course 202. A patient (user 10) navigates the virtual navigation course 202 and the virtual reality system 100 monitors the progress of a user 10 through the virtual navigation course 202. The performance of the user 10 is then determined by using one or more metrics (performance metrics), which will be discussed further below. In this embodiment, these performance metrics are calculated by the virtual reality system 100 and in particular the user system 130 and processor 132, using data received from the sensors 114 and sensors 126. This functional vison assessment may be repeated over time for a user 10 to assess, for example, the progression of his or her eye disease or improvements from a treatment. For such an assessment over time, the performance metrics from each time the user 10 navigates the virtual navigation course 202 are compared against each other.

The virtual navigation course 202 is stored in the non-volatile storage 138, and the processor 132 displays on the integrated display 112 aspects of the virtual navigation course 202 depending upon input received from the sensors 114. Features of the virtual navigation course 202 will be discussed further below. Various features of the virtual reality environment 200 that are rendered by the processor and shown on the integrated display 112 will generally be referred to as “simulated” or “virtual” objects in order to distinguish them from an actual or “physical” object. Likewise, the term “physical” is used herein to describe a non-simulated or non-virtual object. For example, the room of a building in which the user 10 uses the virtual reality system 100 is referred to as a physical room 20 having physical walls 22. In contrast, a room of the virtual reality environment 200 that is rendered by the processor 132 and shown on the integrated display 112 is a simulated room or virtual room 220. In this embodiment, the virtual navigation course 202 approximates an indoor home environment, however, it is not so limited. For example, the virtual reality environment 200 may resemble any suitable environment, including for example, an outdoor environment such as a crosswalk, parking lot, or street.

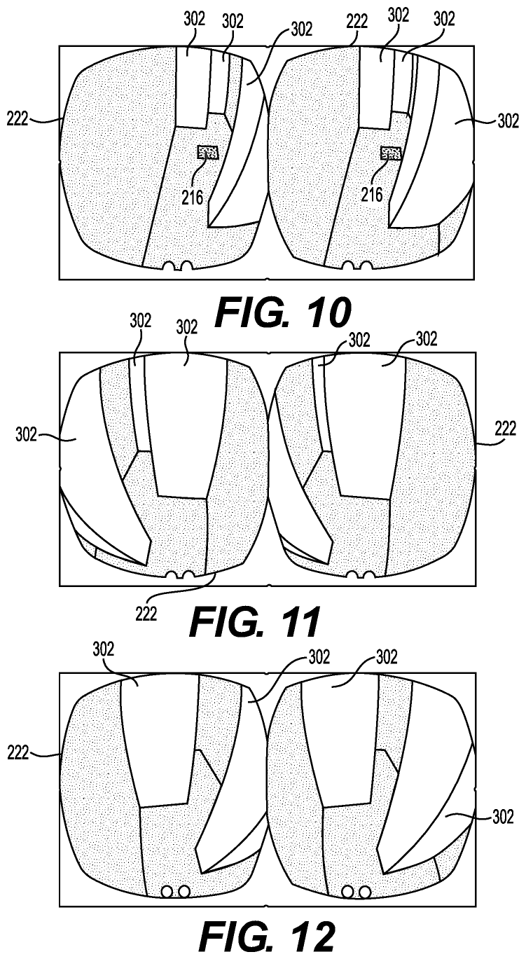

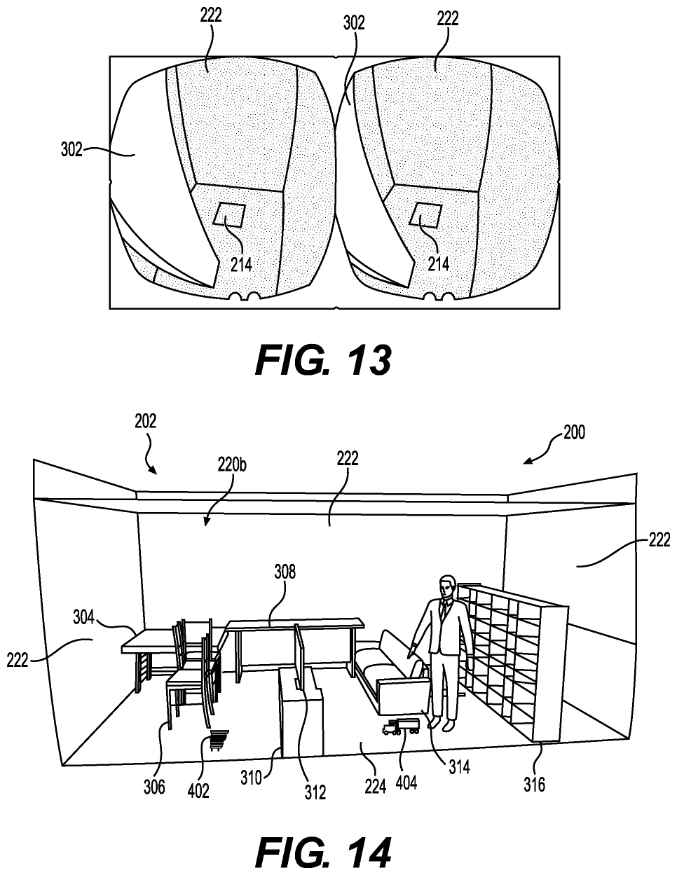

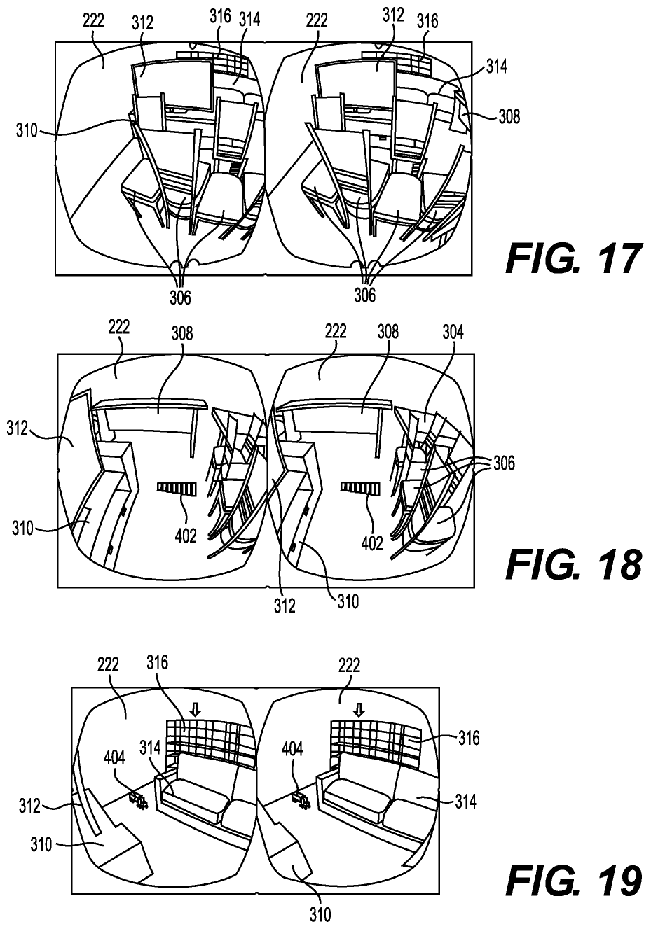

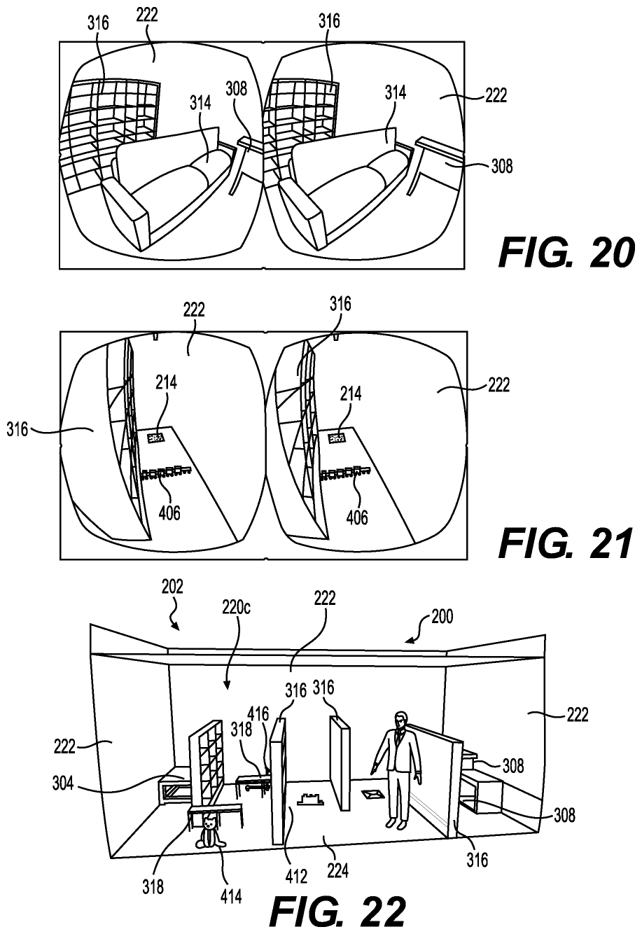

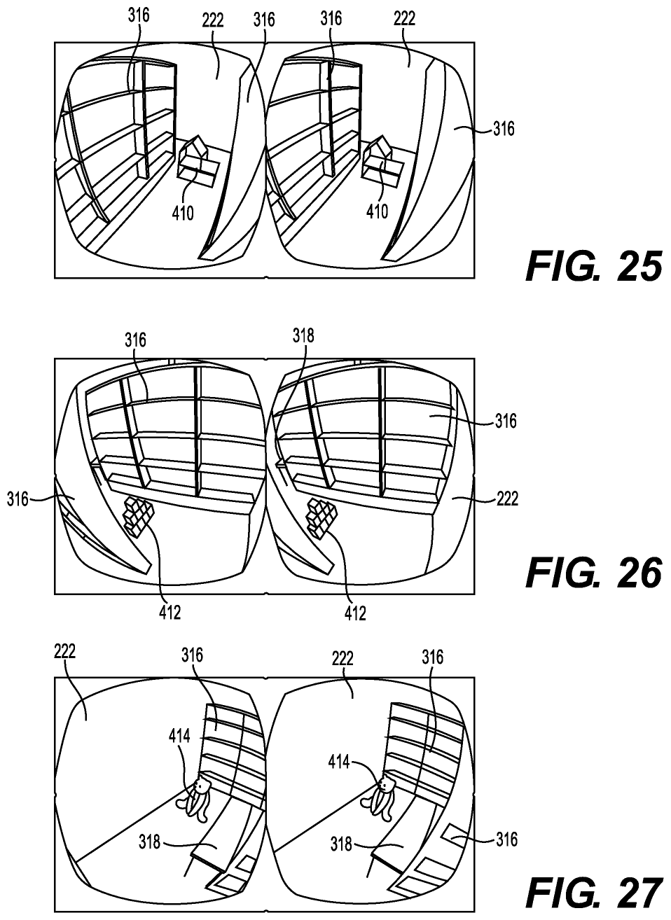

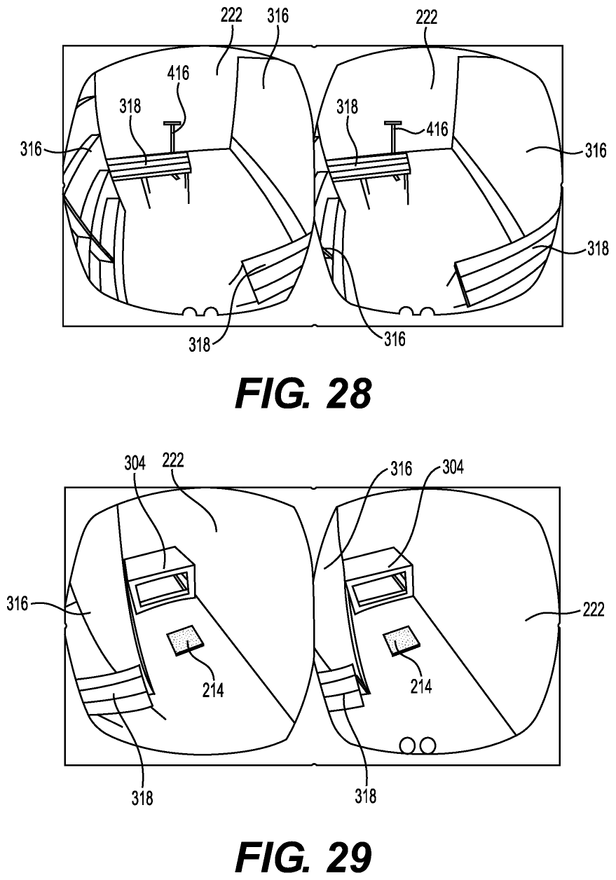

For the functional vision assessment, a patient (user 10) navigates a path 210 through the virtual navigation course 202. The path 210 includes a starting location and an ending location. In this embodiment, the path 210 is set in a simulated room 220 with virtual obstacles. Examples of such virtual rooms are shown in the figures, including a first virtual room 220a (FIGS. 8-13), a second virtual room 220b (FIGS. 14-21), and a third virtual room 220c (FIGS. 22-29). In this embodiment, a portion of the virtual navigation course 202 is located in each virtual room 220 of a plurality of rooms, such as the first virtual room 220a, second virtual room 220b, and third virtual room 220c. As will be described further below, each of virtual room 220 has different attributes. The virtual navigation course 202, however, is not so limited. For example, the virtual navigation course 202 can be a single virtual room 220. When the virtual navigation course 202 is implemented using a single virtual room 220, the various attributes of the virtual navigation course 202 discussed further below, such as different contrast levels or luminance, may be implemented in different sections of the virtual room 220.

In this embodiment, each virtual room 220 includes simulated walls 222 and a virtual floor 224. Each virtual room 220 also includes a start position 212 and an exit 214. The start position 212 of the first virtual room 220a is the starting location of the path 210, and the exit 214 of the last room used in the assessment, which in this embodiment is the third virtual room 220c, is the ending location.

The path 210 and direction the user 10 should take to navigate the path 210 is designed to be readily apparent to the user 10. In many instances, the user 10 has but one way to go, with boundaries of the path 210 being used to direct the user 10. Audio prompts and directions, however, may be programmed into the virtual navigation course 202 such that when the processor 132 identifies that the user 10 has reached a predetermined position in the path 210, the processor 132 plays an audio instruction on speakers (not shown) integrated into the head-mounted display 110.

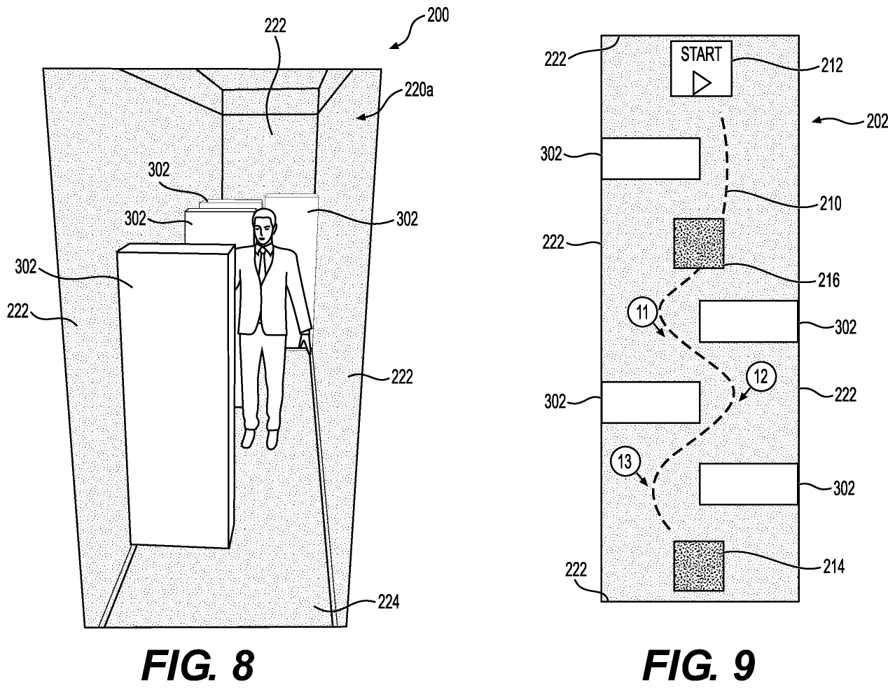

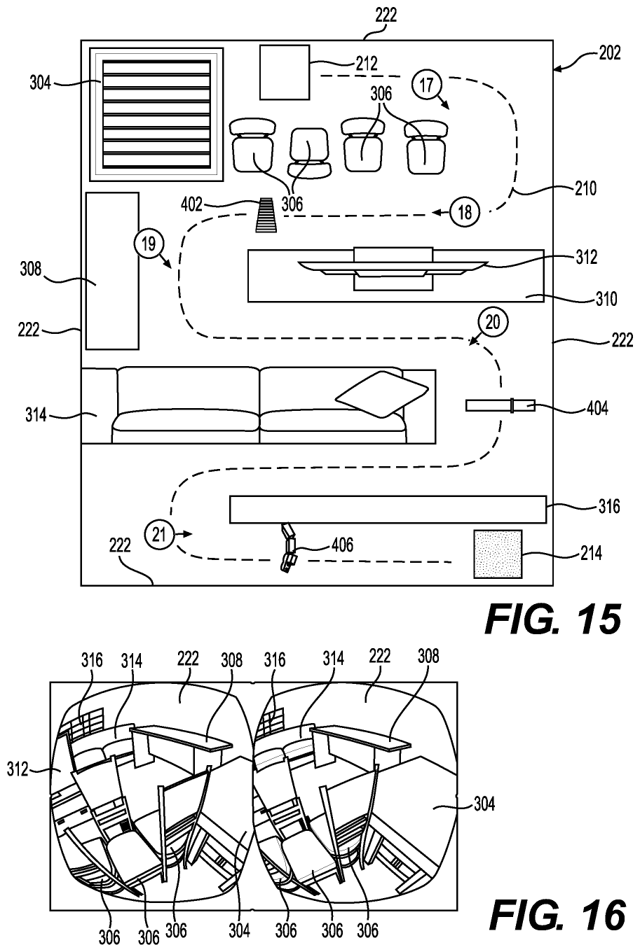

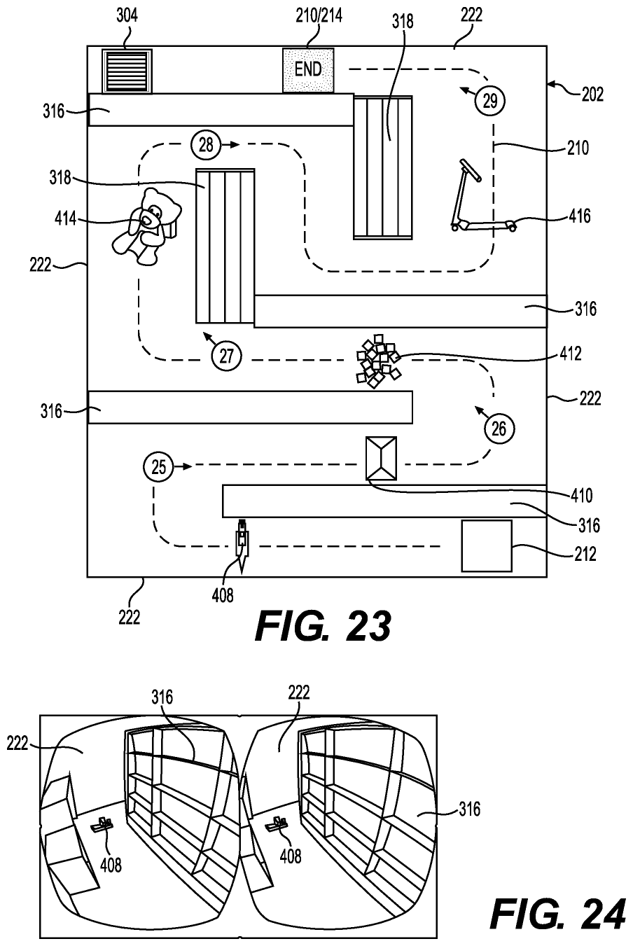

Navigation of the virtual navigation course 202 by a user will now be described with reference to FIGS. 8-29. FIG. 8 is a perspective view of the first virtual room 220a, and FIG. 9 is a plan view of the first virtual room 220a taken from above. FIG. 14 is a perspective view of the second virtual room 220b, and FIG. 15 is a plan view of the second virtual room 220b taken from above. FIG. 22 is a perspective view of the third virtual room 220c, and FIG. 23 is a plan view of the third virtual room 220c taken from above. FIGS. 10-13, 16-21, and 24-29 show what would be displayed on the integrated display 112 of the head-mounted display 110 as the user 10 navigates the virtual navigation course 202. FIGS. 10-13 are views in the first virtual room 220a, FIGS. 16-21 are views in the second virtual room 220b, and FIGS. 24-29 are views in the third virtual room 220c. Unless otherwise indicated, the location of the user 10 in each of the views shown in FIGS. 10-13, 16-21, and 24-29 is indicated in the corresponding plan view for respective the virtual room 220 with a circle surrounding the figure number and an arrow to indicate the direction the user 10 is looking.

As can be seen in FIG. 8, the first virtual room 220a simulates a hallway. In this embodiment, the first virtual room 220a preferably has a width that comfortably allows one individual to walk between a column 302 (discussed further below) located in the first virtual room 220a and the virtual wall 222 of the first virtual room 220a. In this embodiment, the first virtual room 220a preferably has a width of approximately 4 feet. To simulate a hallway, the length of the first virtual room 220a is preferably much greater than the width of the first virtual room 220a. The length of the first virtual room 220a may be preferably at least five times the width of the first virtual room 220a, which in this embodiment is approximately 21 feet.

The path 210, which is shown by the broken line in FIGS. 9, 15, and 23, is defined by the virtual walls 222 of the first virtual room 220a and a plurality of columns 302. In this embodiment, each of the columns 302 has a width of about 1.5 feet and extends from one of the side virtual walls 222 of the first virtual room 220a. This leaves approximately 2.5 feet between the column 302 and the virtual wall 222, which comfortably allows an individual to walk between the column 302 and the virtual wall 222. In this embodiment, an objective of the first virtual room 220a is to provide a suitable room and path 210 for assessing the vision of a user 10 with even very poor vision, such as a user 10 characterized as having light perception only vision. Each column 302 in this embodiment is opaque and has a height that is preferably from 7 feet to 8 feet, such that each column 302 is at least eye level with an average adult as he or she stands (approximately 5 feet) and preferably taller. Beyond the height of each column 302, the columns 302 are made even easier to see in this embodiment by being glowing columns, such that they have a higher brightness than the brightness of the surroundings, which, in this embodiment, is the virtual walls 222 and virtual floor 224 of the first virtual room 220a.

As described below, the user 10 will traverse the path 210 by navigating around each column 302 to reach the checkpoint at the exit 214. After the user stands on the green checkpoint at the exit 214, the virtual room 220 automatically re-configures from the first virtual room 220a to the second virtual room 220b. The user 10 is then instructed to turn around and continue navigating the path 210 in the second virtual room 220a. In other words, the exit 214 of the first virtual room 220a is the start position 212 of the second virtual room 220b. This process is repeated for each virtual room 220 in the virtual navigation course 202. This configuration allows the same physical room 20, such as a 24 foot by 14 foot space, to be used for an infinite number of rooms. The second virtual room 220b and third virtual room 220c are 21 feet by 11 feet, in this embodiment.

When the virtual reality environment 200 is initially loaded and displayed on the integrated display 112, the user is placed at the start position 212 in the first virtual room 220a. FIG. 10 is a view of the integrated display 112 with the user 10 looking toward the first column 302. In this embodiment, the user 10 is located next to the left virtual wall 222 of the first virtual room 220a, and the first column 302 is adjacent to the right virtual wall 222 of the first virtual room 220a. The user 10 proceeds to navigate through the first virtual room 220a by first moving forward past the first column 302 and then weaving past each successive column 302 to the end of the hall (first virtual room 220a) and to the exit 214 of the first virtual room 220a. In this embodiment, the columns 302 are staggered successively down the length of the first virtual room 220a, with the second column 302 being adjacent to the left virtual wall 222, the third column 302 being adjacent to the right virtual wall 222, and the fourth column 302 being adjacent to the left virtual wall 222. The exit 214 in this embodiment is located behind the fourth column 302.

One of the performance metrics used to evaluate the patient's vision and efficacy of any treatment is the time it takes for the user 10 to navigate (traverse) the path 210. In this embodiment, the start position 212 for the first virtual room 220 is the starting position of the path 210 and thus the time is recorded by the virtual reality system 100 when the user 10 starts at the start position 212 of the first virtual room 220a. The time is also recorded when the user 10 reaches various other checkpoints (also referred to as waypoints), such as the exit 214 of each virtual room 220, and the ending location of the path 210, which in this embodiment is the exit 214 of the third virtual room 220c. In this embodiment, the first virtual room 220a includes an intermediate checkpoint 216. Although shown here with only one intermediate checkpoint 216, any suitable number of intermediate checkpoints 216 may be used in each virtual room 220. From these times, the virtual reality system 100 can precisely determine the time it takes for a user 10 to navigate the virtual navigation course 202 and traverse the path 210. When time is recorded for other checkpoints, the time for the user 10 to reach these checkpoints may also be similarly determined.

The virtual reality system 100 also tracks the position, and thus the distance a user travels in completing the virtual navigation course 202 can be calculated. Although the virtual navigation course 202 is designed to be readily apparent to the user 10 and there is an optimal, shortest way to traverse the path 210, a user 10 may deviate from this optimal route. The user 10 may, for example, not realize a turn and travel farther, such as closer to a virtual walls 222 or other virtual object, before making the turn, thus increasing the distance traveled by the user 10 in navigating the virtual navigation course 202. The total distance traveled and/or the deviation from the optimal route may be another performance metric used to evaluate the performance of a user 10 in navigating the virtual navigation course 202.

A further performance metric used to evaluate the performance of a user 10 in navigating the virtual navigation course 202 is the number of times that the user 10 collides with the virtual objects in each virtual room 220. In the first virtual room 220a, the virtual objects with which the user 10 could collide include, for example, the virtual walls 222 and the column 302. In this embodiment, a collision with a virtual object is determined as follows, although any suitable method may be used. The virtual reality system 100 records the precise movement of the head of the user 10 using the sensors 114 for the head-mounted display 110. As discussed above, these sensors 114 report the real-time position of the head of the user 10. From the real-time position of the head of the user 10, the virtual reality system 100 extrapolates the dimensions of the entire body of the user 10 to compute a virtual box around the user 10. When the virtual box contacts or enters a space in the virtual reality environment 200 in which the virtual objects are located, the virtual reality system 100 determines that a collision has occurred and records this occurrence. Additional sensors on (or that detect) other portions of the user 10, such as the feet, shoulders, and hands (e.g., sensors 126 of the controllers 120), may also be used to determine whether a limb or other body part collided with the virtual object. The functional vision assessment of the present embodiment can thus precisely and accurately determine the number of collisions.

Still another performance metric used to evaluate the performance of a user 10 in navigating the virtual navigation course 202 is the amount of the course completed at each luminance level (discussed further below). As discussed above, the path 210 contains a plurality of checkpoints including the exits 214 of each virtual room 220 and any intermediate checkpoints, such as the intermediate checkpoint 216 in the first virtual room 220a. When the user 10 reaches a checkpoint, the virtual reality system 100 records the checkpoints reached by the user 10. If the entire virtual navigation course 202 is too difficult for the user 10 to complete (by becoming stuck and unable to find their way through the path 210 or by hitting too many (predetermined number) virtual objects such as virtual walls 222 and virtual obstacles), the user 10 may complete only portions of the virtual navigation course 202. Comparing between successive navigations of the virtual navigation course 202, such as when evaluating a treatment, for example, the user 10 may be able to complete the same portion of the course faster, or potentially complete additional portions of the course (e.g., reach additional checkpoints). Thus, an advantage of the embodiments described herein is that a single course that can be used for all participants, accommodating the wide range of visual abilities of the patient population, because an individual user 10 does not necessarily have to complete the most difficult portions of the course if they are unable to do so. In contrast, separate physical navigation courses would be required, each with different levels of difficulty, and would need to be able to accommodate the wide range of visual abilities of the patient population.

When the user 10 reaches the exit 214 of the first virtual room 220a, the second virtual room 220b is displayed on the display screen with the user 10 being located in the start position 212 of the second virtual room 220b, as shown in FIG. 16. The second virtual room 220b of this embodiment is shown in FIGS. 14-21. As can be seen in FIG. 14, the second virtual room 220b simulates a larger room than the first virtual room 220a, which is wider in this embodiment (as discussed above 21 feet by 11 feet). In this embodiment, the second virtual room 220b includes virtual obstacles around which the user 10 must navigate. In the first virtual room 220a the virtual obstacles are the columns 320, but in the second virtual room 220b the virtual obstacles are virtual furniture. The second virtual room 220b thus includes a plurality of virtual furniture. The virtual furniture in this embodiment is preferably common household furniture, including, for example, at least one of a chair, a table, a bookcase, a bench, a sofa, and a television. In this embodiment, the virtual furniture includes a square table 304, similar to a dining room table; chairs 306, similar to dining chairs; an elongated rectangular table 308; a media console 310 with a flat panel television 312 located thereon; a sofa 314; and a bookcase 316. As with the column 302 in the first virtual room 220a, pieces of the virtual furniture are arranged adjacent to the virtual walls 222 and to each other to create the path 210 for the user 10 to traverse. The user 10 navigates the second virtual room 220b of the virtual navigation course 202 by moving around the arrangement of virtual furniture from the start position 212 to the exit 214, and the virtual reality system 100 evaluates the performance of the user 10 using the performance metrics discussed herein. Although the virtual obstacles (virtual furniture) are discussed as being arranged to have the user 10 navigate around them, the arrangement of the virtual obstacles (virtual furniture) is not so limited and may also be arranged, for example and without limitation, such that the user 10 has to go underneath (crouch and move underneath) a virtual obstacle or step over virtual obstacles.

The plurality of virtual furniture in the second virtual room 220b has a plurality of heights and sizes. The bookcase 316, for example, preferably has a height of at least 5 feet. Other virtual furniture has lower heights; for example, the square table 304 and media console 310 each have a height between 18 inches and 36 inches.

In the second virtual room 220b, the virtual navigation course 202 also includes a plurality of virtual obstacles that can be removed (referred to hereinafter as removable virtual obstacles). In this embodiment, the removable virtual obstacles are located in the path 210 and are toys located on a virtual floor 224 of the second virtual room 220b. The removeable virtual obstacles are preferably designed to have a lower height than the virtual furniture used to define the boundaries of the path 210. The user 10 is instructed to remove the obstacles as they are encountered along the path. If the user 10 does not remove the removable virtual obstacle, the user 10 may collide with the obstacle and the collision may be determined as discussed above for collisions with the virtual furniture. The number of collisions with the removeable virtual obstacles is another example of a performance metric used to evaluate the performance of the user 10 and may be evaluated separately or together with the number of collisions with the virtual furniture or other boundaries of the path 210.

The removeable virtual obstacles are preferably objects that could be found in a walking path in the real world and in this embodiment are preferably toys, but the removeable virtual obstacles are not so limited and may include other items such as colored balls, colored squares, and other items commonly found in a household (e.g., vases and the like). Toys may be particularly preferred as potential users 10 include children (pediatric patients) that have toys in their own household. Additionally, it is reasonable to expect that many users are familiar with and would reasonably expect toys to be in a walking path as many users have children and/or grandchildren. In this embodiment, the removeable virtual obstacles include a multicolored toy xylophone 402, a toy truck 404, and a toy train 406. In this embodiment, the removeable virtual obstacles are located on the virtual floor 224, but they are not so limited. Instead, for example and without limitation, the removeable virtual obstacles may appear to be floating, that is they are positioned at approximately eye level (about 5 feet for adult users 10 and lower, such as 2.5 feet for users 10 who are children) within the path 210. The virtual reality system 100 may use the sensors 114 of the head-mounted display 110 to determine the head height of the user 10 and then place the removeable virtual obstacles at head height for the user, for example. The removeable virtual obstacles also may randomly appear in the path 210.

Any suitable method may be used to remove the virtual obstacles. In this embodiment, the removeable virtual obstacles may be removed by the user 10 looking directly at a virtual obstacle. The user 10 may move his or her head so that the virtual obstacle is located approximately in the center of his or her field of view, such as in the center of the integrated display 112, and holding that position (dwelling) for a predetermined period of time. The virtual reality system 100 then removes the virtual obstacle from the virtual reality environment 200. When the virtual reality system 100 includes a controller 120, the virtual reality system 100 may remove the virtual obstacle from the virtual reality environment 200 in response to a user input received from a user input on the controller 120. For example, the user 10 can press a button 122 on the controller 120 with the virtual obstacle in the center of his or her field of view, and in response to the input received from the button 122 the virtual reality system 100 removes the virtual obstacle.

When the user 10 reaches the exit 214 of the second virtual room 220b, the third virtual room 220c is displayed on the display screen with the user 10 being located in the start position 212 of the third virtual room 220c, as shown in FIG. 24. The third virtual room 220c of this embodiment is shown in FIGS. 22-29. As can be seen in FIG. 22, the third virtual room 220c is similar to the second virtual room 220b and includes virtual furniture of different heights. The virtual furniture in the third virtual room 220c includes a square table 304, bookcases 316, and benches 318. The third virtual room 220c also includes virtual obstacles. The removeable virtual obstacles in the third virtual room 220c, like the removeable virtual obstacles in the second virtual room 220b, are toys. The toys in the third virtual room 220c include a toy ship 408, a dollhouse 410, a pile of blocks 412, a large stuffed teddy bear 414, and a scooter 416. The vertical furniture is arranged such that the path 210 taken through the third virtual room 220c is different from the path 210 through the second virtual room 220b. These differences may include that the portion of the path 210 in the third virtual room 220c is longer than the portion of the path 210 in second virtual room 220b and that the portion of the path 210 in the third virtual room 220c is has more turns than the portion of the path 210 in second virtual room 220b.

In this embodiment, the second virtual room 220b and the third virtual room 220c have different contrasts. The second virtual room 220b is a high-contrast room where the virtual obstacles, have a high contrast with their surroundings. In this embodiment, the backgrounds, such as the virtual walls 222 and virtual floor 224, have a light color (light tan, in this embodiment), and the virtual obstacles have dark or vibrant colors. Similarly, the removable virtual obstacles of this embodiment are brightly colored children's toys, which stand out from the light, neutral-colored background. On the other hand, the third virtual room 220c is a low-contrast room in which the virtual obstacles, have coloring similar to that of the background. For example, the virtual obstacles, may be white or gray in color with the background being a light tan or white. With the low-contrast room located after the high-contrast room, the virtual navigation course 202 of this embodiment is progressively more difficult.

The placement of the virtual objects, their color, light intensity, and other physical attributes, thus may be strategized to test for specific visual functions. With color, for example, the objects in the second virtual room 220b are all dark colored having high contrast with the white walls, and in the third virtual room 220c, all of the objects are white or gray having low contrast with the white walls and white floor. This increases the difficulty of the third virtual room 220c for participants that have trouble with contrast sensitivity (a specific visual function). In another example of light intensity, the columns 302 in the third virtual room 220c are glowing to make them possible to see for patients with severe vision loss (e.g. light perception vision).

The functional vision assessment may be performed under a plurality of different environmental conditions. In a preferred embodiment of the invention, a user 10 navigates the virtual navigation course 202 under one environmental condition and then navigates the virtual navigation course 202 at least one other time with a change in the environmental condition. Instead of repeating the virtual navigation course 202 under different environmental conditions, this assessment may also be implemented by virtual rooms of virtual navigation course 202 with each room of the virtual navigation course 202 having the changed environmental condition.

One such environmental condition is the luminance of the virtual reality environment 200. In one preferred embodiment, the user 10 may navigate the virtual navigation course 202 a plurality of times in a single evaluation period, and with each navigation of the course, the virtual reality environment 200 has a different luminance. For example, the user 10 may navigate the virtual navigation course 202 the first time with the lowest luminance value of 0.1 cd/m². The virtual navigation course 202 is then repeated with a brighter luminance value of 0.3 cd/m², for example. Then, the user 10 navigates the course a third time, with another brighter luminance value of 1 cd/m2, for example. In this embodiment, the user 10 navigates the virtual navigation course 202 multiple time each at sequentially brighter luminance value between 0.1 cd/m2 and 100 cd/m2. The luminance values are equally spaced (½ log between each light level) and thus the luminance values are 0.5 cd/m2 (similar to the light level on a clear night with a full moon), 1 cd/m2 (similar to twilight), 2 cd/m2 (similar to minimum security risk lighting), 5 cd/m2 (typical lighting level for lighting on the side of the road), 10 cd/m2 (similar to sunset), 20 cd/m2 (similar to a very dark, overcast day), 50 cd/m2 (similar to the lighting of a passageway or outside working area), and 100 cd/m2 (similar to the lighting in a kitchen). To navigate at the lowest luminance values, the user 10 undergoes about 20 minutes of dark adaptation before starting the test, so that the eyes of the user 10 can adjust to the dark and allow them the best chance possible to be able to navigate the virtual navigation course 202 at the lowest light level. It is thus advantageous to begin the test at the lowest luminance value and sequentially increase the luminance value. This approach also helps to standardize and effectively compare results between different evaluation periods.

One of the performance metrics used may include the lowest luminance value passed. For example, a user may not be able to complete the virtual navigation course 202 at one level, by becoming stuck and unable to find their way through the path 210 or by hitting too many virtual objects such as virtual walls 222 and virtual obstacles. Completing the virtual navigation course 202 at a certain luminance level or having a number of collisions lower than a predetermined value may be considered passing the luminance value.

The head-mounted display 110 may be equipped with eye tracking (an eye tracking enabled device). The virtual reality system 100 could collect data on the position of the eye, which could be used for further analysis. This eye tracking data may be a further performance metric.

As discussed above, the functional vision assessment discussed herein can be used to assess the progress of a patient's disease or treatment over time. The user 10 navigates the virtual navigation course 202 a first time and then after a period of time, such as days or months, the user 10 navigates the virtual navigation course 202 again. The performance metrics of the first navigation can then be compared to the subsequent navigation as an indication of how the disease or treatment is progressing over time. Additional further navigations of the virtual navigation course 202 can then be used to further assess the disease or treatment over time.

With repeated navigation of the virtual navigation course 202, there is a risk that the user 10 may start to “learn” the course. For example, the user 10 may remember the location of the virtual obstacles and thus the virtual navigation course 202 loses its effectiveness as an assessment tool. To avoid this, one of a plurality of unique course configurations (16 unique course configurations in this embodiment, for example) are selected at random at the start of the assessment. Between each of the plurality of unique course configurations, the total length of the path 210 is kept the same, as is the number of left/right turns and virtual obstacles during randomization. The position of the virtual obstacles and the order in which they appear also may be changed between each of the plurality of unique course configurations. Likewise, the position and orientation of the various virtual furniture also may be changed between each of the plurality of unique course configurations.

As described above, the environmental conditions, such as luminance, and the contrast is static. The luminance level is set at the same level for all three virtual rooms 220. Likewise, the contrast is generally the same within each of the first virtual room 220a, second virtual room 220b, and third virtual room 220c. The invention, however, is not so limited and other approaches could be taken, including, for example, making the environmental conditions dynamic. For example, either one or both of the luminance level and contrast could be dynamic, such that either parameter increases or decreases in a continuous fashion as the user navigates the virtual navigation course 202.

A preferred implementation of the functional vision assessment is described as follows. In this embodiment, the functional vision assessment using the virtual navigation course 202 involves a 20-minute period of dark adaptation before the user 10 attempts to navigate the virtual navigation course 202 at increasing levels of luminance. When the user 10 completes the virtual navigation course 202 (or is unable to continue navigating the virtual navigation course 202), a technician may ensure the participant is correctly aligned before moving on to the next luminance level. With a click of a button, a new course configuration is randomly chosen from the 16 unique course configurations with the same number of turns and/or obstacles.

The base course configuration for the virtual navigation course 202 is, as described in more detail above, designed with a series of three virtual rooms 220 (first virtual room 220a, second virtual room 220b, third virtual room 220c) and four checkpoints (the exit 214 of each virtual room 220 and intermediate checkpoint 216) that permit the participant (user 10) to complete only a portion of the virtual navigation course 202, if the remainder of the virtual navigation course 202 is too difficult to navigate. The first virtual room 220a, which may be referred to herein as the Glowing Column Hallway, is designed to simulate a hallway with dark virtual walls 222 and virtual floor 224 and four tall columns 302. As the luminance (cd/m2) level increases, the luminance emitting from the column 302 increases. The Glowing Column Hallway is the easiest of the three column 302 to navigate and may be designed for participants with severe vision loss (e.g., Light Perception only or LP vision). The second virtual room 220b, herein referred to as the High Contrast Room, is a 21-foot by 11-foot room with light virtual walls 222 and virtual floor 224 and dark colored virtual furniture (virtual obstacles) that delineates the path 210 the participant (user 10) should traverse. At various points along the path, there are brightly colored virtual toys (removeable virtual obstacles) obstructing the path 210 that can be removed if the participant looks directly at the toy and presses a button 122 on the controller 120 in their hand. The third virtual room 220c, herein referred to as the Low Contrast Room, is similar to the High Contrast Room (second virtual room 220b), but there are an increased number of turns, increased overall length, and the all of the objects (both virtual furniture and virtual toys) are white and/or grey, providing very low contrast with the virtual walls 222 and virtual floor 224 in the third virtual room 220c.

A study was conducted to assess the reliability and construct validity of the virtual navigation course 202. This study was conducted using 30 healthy volunteers, having approximately 20/20 vision or vision that is corrected to approximately 20/20 vision. The study participants ranged in age from 25 years old to 44 years old. Forty percent of them were female and 57% wore glasses or contacts.

The study was conducted over 3 weeks. Each participant (user 10) was tested five times. In the first and second weeks, the participant (user 10) conducted a test and a retest, and in the third week, the third week the participant (user 10) conducted a single test. Each test or retest comprised the user 10 navigating the path 210 of the virtual navigation course 202 discussed above three different times. The environmental condition of luminance level was changed between each of the three times the user 10 navigated the path 210. The first time the user 10 traversed the path 210 the luminance level was set at 1 cd/m2. The second time the user 10 traversed the path 210 the luminance level was set at 8 cd/m2. And, the third time the user 10 traversed the path 210 the luminance level was set at 100 cd/m2.

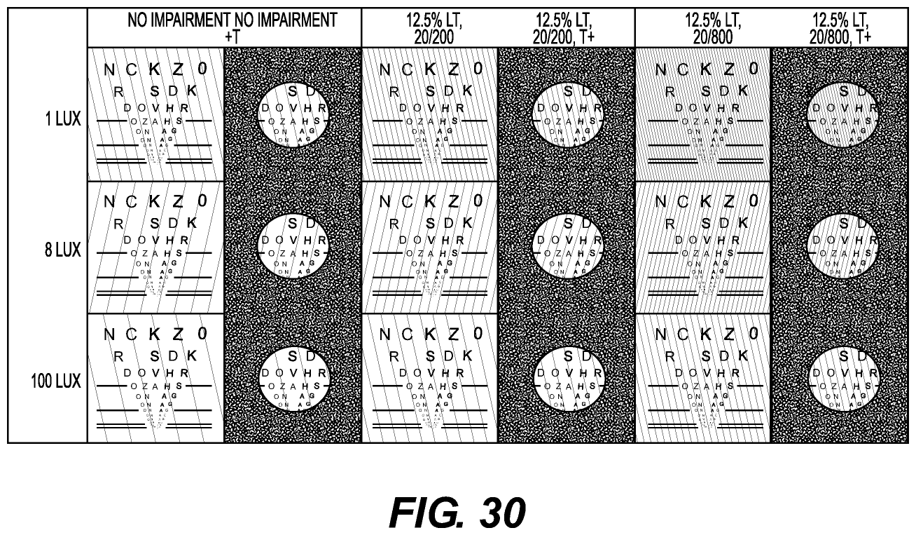

Some of the participants conducted each test under simulated visual impairment conditions. FIG. 30 illustrates the simulated impairment conditions used in this study. Three different impairment conditions were simulated in this study and each of the three impairment conditions had two permutations for a total of six different impairment conditions. The three different impairment conditions were no impairment (20/20 vision), 20/200 vision with light transmittance (“LT” in FIG. 30) reduced by 12.5%, and 20/800 vision with light transmittance reduced by 12.5%. Some participants having each of these three impairment conditions also were also given 30-degree tunnel vision (T+ in FIG. 30). Tunnel vision and reduced light transmittance was used to mimic rod dysfunction.

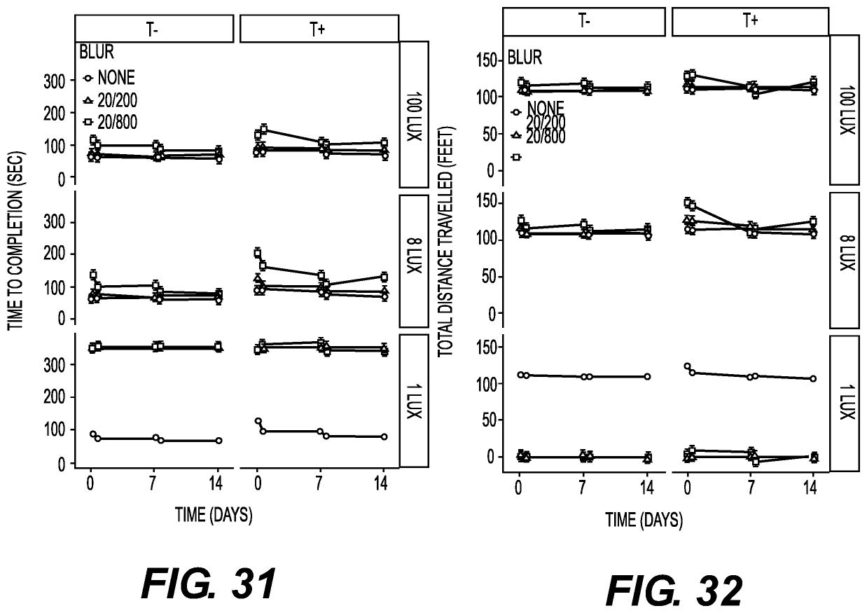

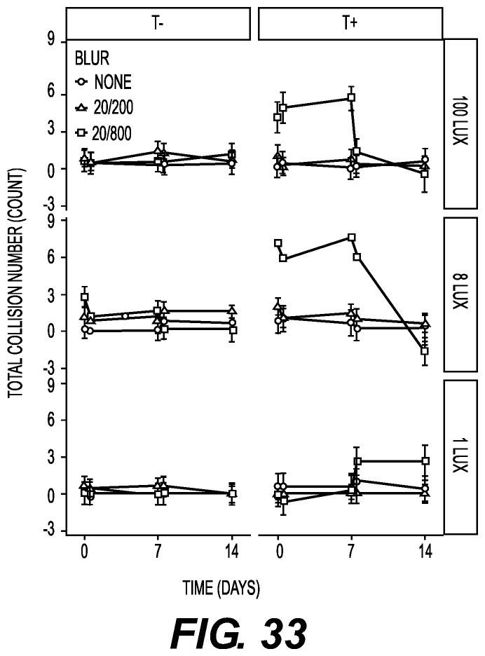

The performance metrics evaluated in this study included the lowest luminance level passed (measured in cd/m2), the time to complete the virtual navigation course 202, the number of virtual obstacles hit, and the total distance traveled. FIG. 31 shows the least squares mean (LSMean) time to complete the virtual navigation course 202 of all participants for a given impairment condition for each test and retest at the different luminance levels. FIG. 32 shows the LSMean total distance traveled of all participants for a given impairment condition for each test and retest at the different luminance levels. FIG. 33 shows the LSMean number of collisions with virtual objects of all participants for a given impairment condition for each test and retest at the different luminance levels.

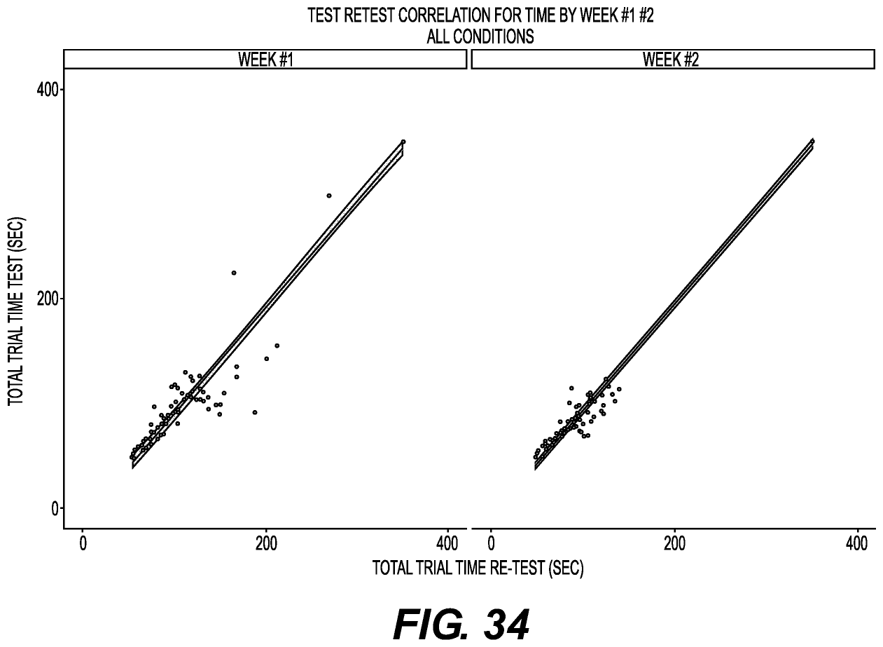

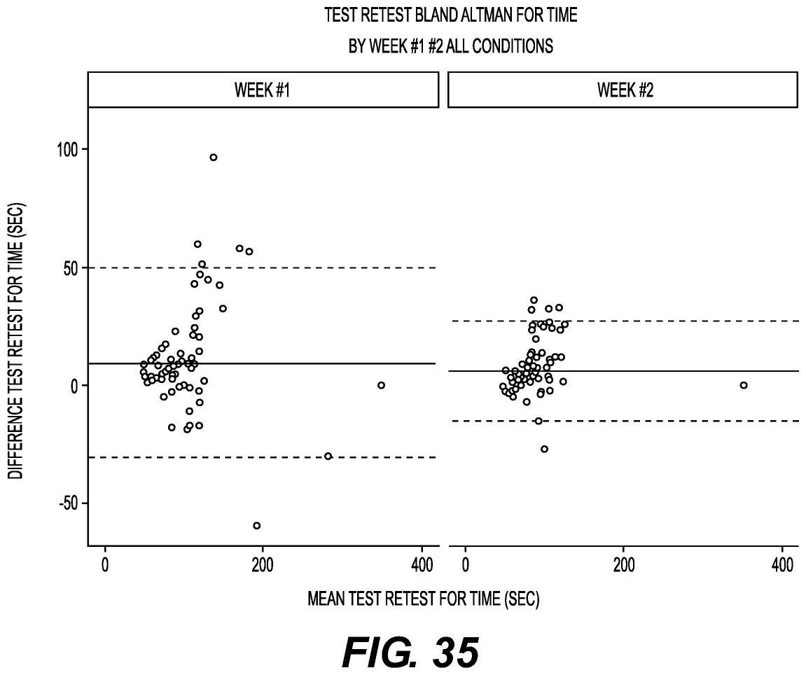

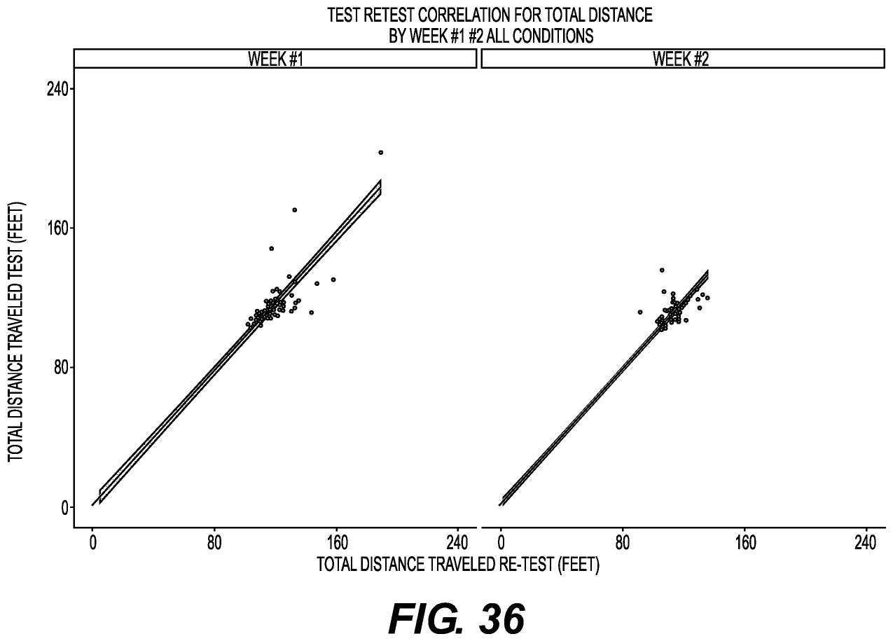

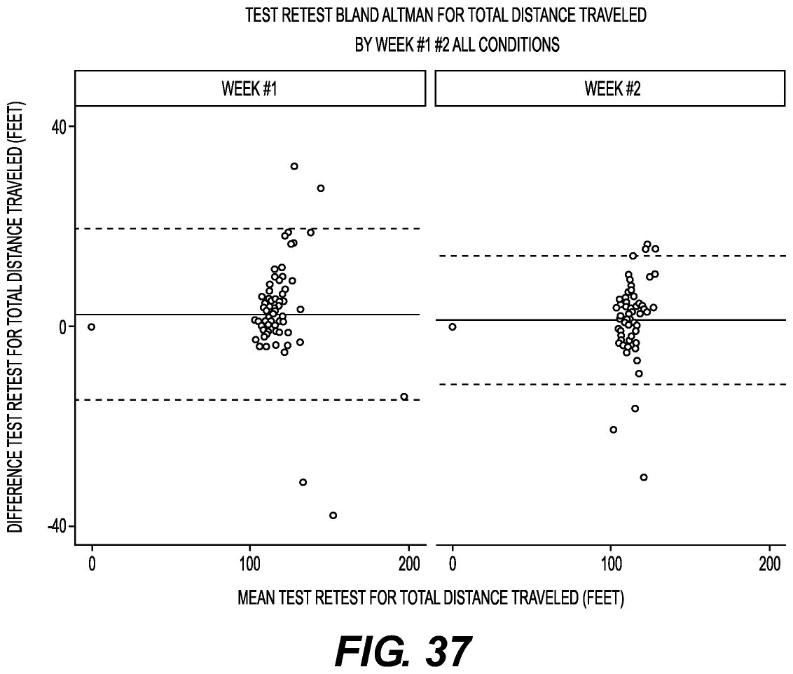

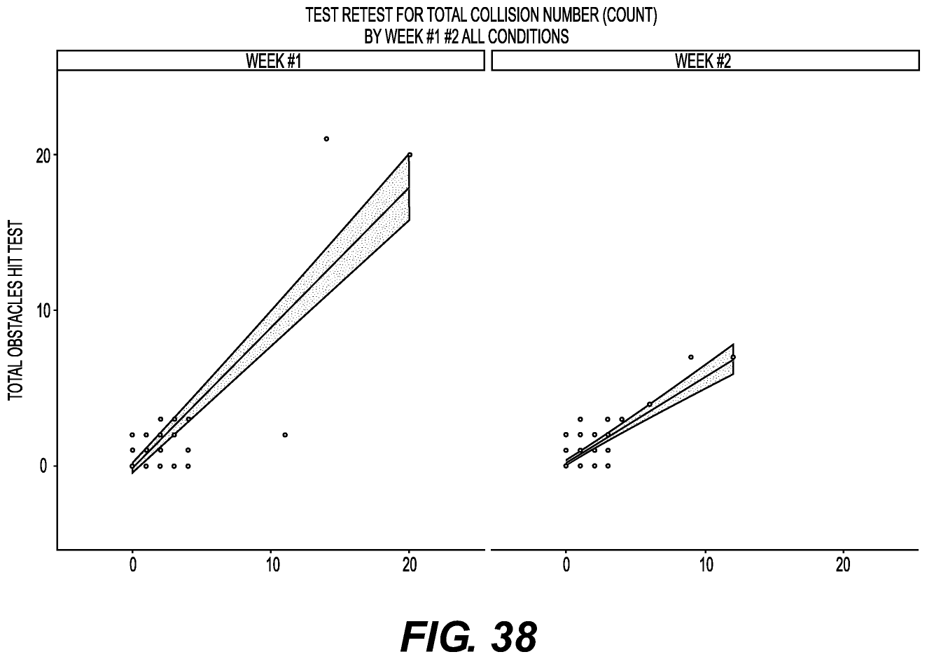

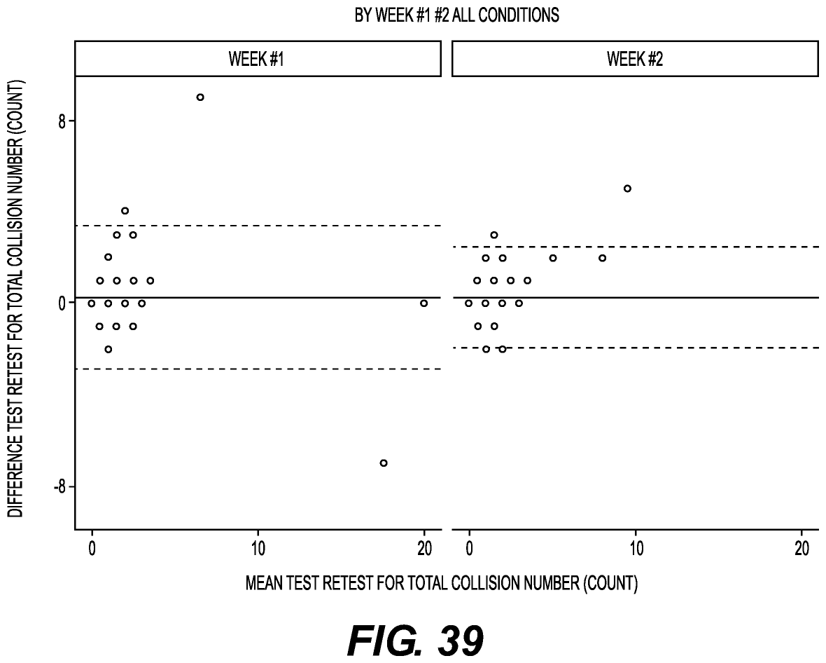

FIGS. 34-39 compare the initial test in each of weeks one and two with the retest in those weeks. FIGS. 34, 36, and 38 are scatter plots, and FIGS. 35, 37, and 39 are Bland-Altman plots. In FIGS. 34, 36, and 38, the mean performance metric taken from all participants within a given impairment condition and luminance level is plotted. FIGS. 34 and 35 evaluate the time to complete the virtual navigation course 202. FIGS. 36 and 37 evaluate the total distance traveled. FIGS. 38 and 39 evaluate the number of collisions with virtual objects.

The study showed that no significant test-retest differences, after applying the Hochberg multiplicity correction, were detected for each performance metric when considered by within the week, luminance level, and impairment condition, with two exceptions. There were test-retest differences detected for the two groups with the worst impairment at the middle luminance level (8 cd/m2) for the first week only. As can be seen in FIG. 31, participants with 20/200 vision, 12.5% light transmittance and tunnel vision demonstrated a test-retest difference (p=0.024) at 8 cd/m2, and participants with 20/800 vision, 12.5% light transmittance and tunnel vision demonstrated a test-retest difference (p=0.004) at 8 cd/m2. As shown in FIG. 35, the mean percent difference in time to complete the virtual navigation course 202 was about 5%. As shown in FIG. 37, the mean percent difference in total distance traded was about 2%. As shown in FIG. 39, the mean percent difference in the number of collisions with virtual objects was about 25%.

The study showed that there are many significant differences detected between groups with simulated visual impairment for the time to complete the virtual navigation course 202 and most of these differences are detected at the lowest luminance levels (1 cd/m2 and 8 cd/m2), as shown in FIG. 31. The study also showed that there are some statistically significant differences in total distance travelled between groups, as shown in FIG. 32. The study further shows that there are significant increases in the number of collisions detected for the group with the most severe simulated impairment condition, as shown in FIG. 33. In the study, the participants with 20/200 vision with 12.5% light transmittance and the participants with 20/800 vision with 12.5% light transmittance were not able to complete the virtual navigation course 202 at the lowest luminance level (1 cd/m2).

### Additional Vision Assessments

The virtual reality system 100 discussed herein may be used for additional vision assessments beyond the functional vision assessment using the virtual navigation course 202. Unless otherwise stated, each of the vision assessments described in the following sections uses the virtual reality system 100 discussed above, and features of one virtual reality environment 200 described herein may be applicable the other virtual reality environments 200 described herein. Where a feature or a component in the following vision assessments is the same or similar to those discussed above, the same reference numeral will be used for these features and components and a detailed description will be omitted.

### Low Vision Visual Acuity Assessment

Many visual acuity assessments use a standard eye chart, such as the Early Treatment Diabetic Retinopathy Study (“ETDRS”) chart. However, patients with very low vision, such as patients from No Light Perception (NLP) to 20/800 vision, are unable to read the letters of the ETDRS chart. Existing methods for assessing the visual acuity of these patients have poor granularity. Such methods typically use different letter sizes at discrete intervals. For patients with very low vision, these intervals are large (having, for example a LogMAR value of 0.2 between the letter sizes). There is thus a large unmet need in clinical trials for a low vision visual acuity assessment with more granular scoring than those available on the market. The low vision visual acuity test (low vision visual acuity assessment) of this embodiment uses the virtual reality system 100 and a virtual reality environment 500 that allows for higher resolution scoring of patients with very low vision.

In the virtual reality environment 500 of this embodiment, the user 10 is presented with virtual object having a high contrast with the background. In this embodiment the virtual objects are black and the background (such as virtual walls 222 and/or virtual floor 224 of the virtual room 220) is white or another light color. The black virtual objects of this embodiment change size or change the virtual distance from the user 10. In this embodiment of the low vision visual acuity test, the user 10 is asked to complete two different tasks. The first task is referred to herein as the Letter Orientation Discrimination Task and the second task is referred to herein as the Grating Resolution Task. In some cases, the user 10 may be unable to complete the Grating Resolution Task. In such a case, the user 10 will be asked complete an alternative second task (a third task) which is referred to herein as the Light Perception Task.

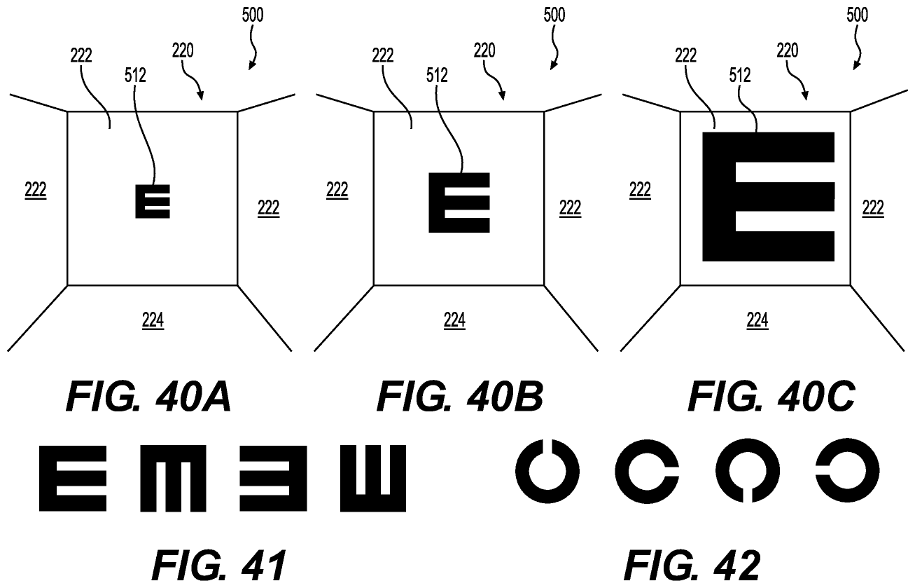

The virtual reality environment 500 for Letter Orientation Discrimination Task is shown in FIGS. 40A-40C. As shown in FIG. 40A, an alphanumeric character 512 is displayed in the virtual room 220. In this embodiment, the alphanumeric characters 512 are capital letters, such as the E shown in FIGS. 40A-41 or the C shown in FIG. 42, for example. The center of the alphanumeric character 512 is approximately eye height. The user 10 is tasked with determining the direction the letter is facing. The alphanumeric character 512 appears in the virtual reality environment 500, having an initial size and then increases in size in a continuous manner. FIG. 40A is, for example, the initial size of the alphanumeric character 512 which then increases in size to, for example, the size shown in FIG. 40B (a medium size) or even the size shown in FIG. 40C (the largest size). Once the user 10 can determine the direction the letter is facing, the user 10 points in the direction that the letter is facing and, in this embodiment, also clicks a button 122 of the controller 120.

The sensors 114 and/or sensors 126 of the virtual reality system 100 identify the direction that the user 10 is pointing and the virtual reality system 100 records the size of the letter in response to input received from the button 122 of the controller 120, when pressed by the user 10. In this embodiment, the performance metrics for the Letter Orientation Discrimination Task are related to the size of the alphanumeric character 512. Such performance metrics may thus include minimum angle of resolution measurements for the alphanumeric character 512, such as MAR and LogMAR. MAR and LogMAR may be calculated using standard methods such as those described by Kalloniatis, Michael and Luu, Charles the chapter on “Visual Acuity” from Webvision (Moran Eye Center, Jun. 5, 2007, available at https://webvision.med.utah.edu/book/part-viii-psychophysics-of-vision/visual-acuity/(last accessed Feb. 20, 2020)), the disclosure of which is incorporated by reference herein in its entirety.

The alphanumeric character 512 may appear in one of a plurality of different directions. In this embodiment, there are four possible directions the alphanumeric character 512 may be facing. These directions are described herein relative to the direction the user 10 would point. FIG. 41 shows the four directions the letter E may face when used as the alphanumeric character 512 in this embodiment. From left to right those directions are: right; down; left; and up. FIG. 42 shows the four directions the letter C may face when used as the alphanumeric character 512 in this embodiment. From left to right those directions are: up; right; down; and left.

For the low vision visual acuity test of this embodiment, the Letter Orientation Discrimination Task is repeated a plurality of times. Each time the Letter Orientation Discrimination Task is repeated one alphanumeric character 512 from a plurality of alphanumeric characters 512 is randomly chosen, and the alphanumeric character 512 direction the alphanumeric character 512 faces is also randomly chosen from one of the plurality of directions. In the embodiment, described above the alphanumeric character 512 appears to at a fixed distance from the user 10 in the virtual reality environment 500 and gradually and continuously gets larger. In alternative embodiments, the alphanumeric character 512 could appear to get closer to the user 10 by either automatically and continuously moving toward the user 10 or the user 10 walking/navigating toward the alphanumeric character 512 in the virtual reality environment 500.

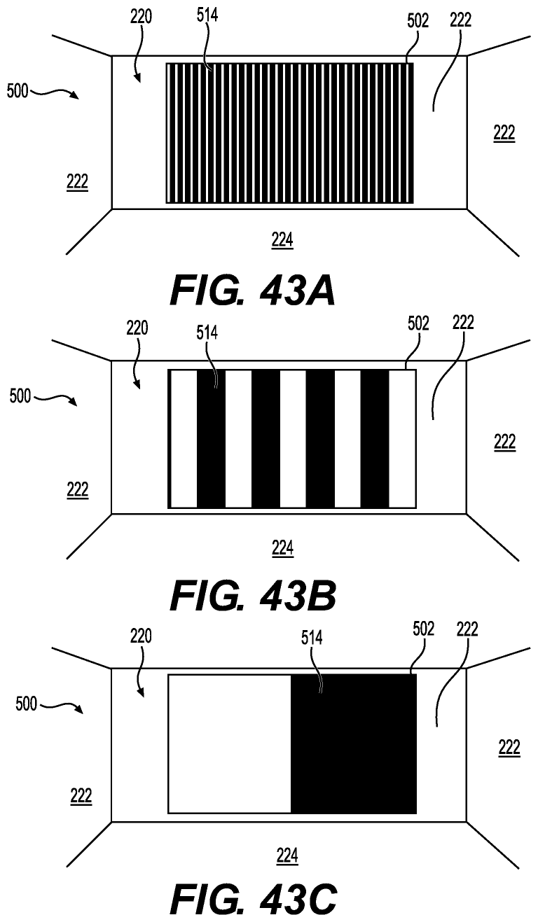

Next, the user 10 is asked to complete the Grating Resolution Task. The virtual reality environment 500 for Grating Resolution Task is shown in FIGS. 43A-43C. In the Grating Resolution Task, a large virtual screen 502 is located on a virtual wall 222 of the virtual room 220. In this embodiment, the virtual screen 502 may resemble a virtual movie theater screen. In the Grating Resolution Task one grating 514 of a plurality of gratings is presented on the virtual screen 502. In this embodiment, the grating 514 is either vertical or horizontal bars. The bars in the grating are of equal widths and alternate between black and white. FIGS. 43A-43C, show an example of the grating 514 with vertical bars.

The grating 514 appears in the virtual reality environment 500 on the virtual screen 502 with each bar having an initial width. The width of each bar in the grating 514 then increases in size in a continuous manner (as the width increases the number of bars decrease). FIG. 43A is, for example, the initial width of bars of the grating 514 which then increases in width to, for example, the width shown in FIG. 43B (a medium width) or even the width shown in FIG. 43C (the largest width having one of each black bar and white bar). Once the user 10 can determine the direction the grating 514 is facing, the user 10 points in the direction that the grating 514 is facing and, in this embodiment, also clicks a button 122 of the controller 120. The sensors 114 and/or sensors 126 of the virtual reality system 100 identify the direction that the user 10 is pointing and the virtual reality system 100 records the width of the bars in the grating 514 in response to input received from the button 122 of the controller 120, when pressed by the user 10. For example, the user 10 would point up or down for vertical bars and left or right for horizontal bars. The performance of the user 10 for the Grating Resolution Task may also be measured using a performance metric based on the width of the bar when the user 10 correctly identifies the direction. As with the Letter Orientation Discrimination Task, the width of the bar may be calculated and reported with MAR and LogMAR, as discussed above.

As with the Letter Orientation Discrimination Task, for the low vision visual acuity test of this embodiment, the Grating Resolution Task may be repeated a plurality of times. Each time the Grating Resolution Task one grating 514 from a plurality of grating 514 is randomly chosen and displayed on the virtual screen 502.

If the participant is unable to complete the Grating Resolution Task, a Light Perception Task will be performed. In this task, the integrated display 112 of the head mounted display 110 will display a completely white light with 100% brightness. The completely white light will be displayed after a predetermined amount of time. The predetermined amount of time will be selected from a plurality of predetermined amount of time, such as randomly selecting a time between 1-15 seconds. The participant is instructed to click the button 122 of the controller 120 when they can see the light. In response to an input received from the button 122 of the controller 120 the virtual reality system 100 determines the amount of time between when the input is received (user 10 presses the button 122) and when the light was displayed on the integrated display 112. In this embodiment the brightness 100%, but the invention is not so limited and in other embodiments, the brightness of the light displayed on the integrated display 112 may be varied.

Although the three tasks are described as part of the same test, in this embodiment each of the tasks may be used individually or in different combinations to provide a low-vision visual acuity assessment.

### Visual Acuity Assessment

The low-vision visual acuity assessment discussed is designed for patients with very low vision, where standard eye charts are not sufficient. Visual acuity assessment for other patients using the Early Treatment Diabetic Retinopathy Study (ETDRS) protocol may also benefit from using the virtual reality system 100 discussed herein. As discussed above, the virtual reality system 100 discussed herein, allows standardized lighting conditions for visual assessments, at a wide variety of locations including home, that is not otherwise suitable for the assessment. The virtual reality system 100 discussed herein could allow for remote assessment of visual acuity, such as at home under standardized lighting conditions.

In the virtual reality environment 520 of this embodiment, the user 10 is presented with a virtual eye chart 522 on a virtual wall 222 of a virtual room 220. The eye chart 522 may be any suitable eye chart, including for example the eye chart using the ETDRS protocol. Although the eye chart 522 is not so limited, and any suitable alphanumeric and symbol/image-based eye charts may be utilized. They eye chart includes a plurality of lines of alphanumeric characters. Each line of alphanumeric characters having at least one alphanumeric character. The alphanumeric characters in a first line of alphanumeric characters 524 are a different size than the alphanumeric characters in a second line of alphanumeric characters 526. When, for example, symbol/image-based eye charts are used, each line includes at least one character (image or symbol) and characters in a first line are a different size than the characters in a second line.

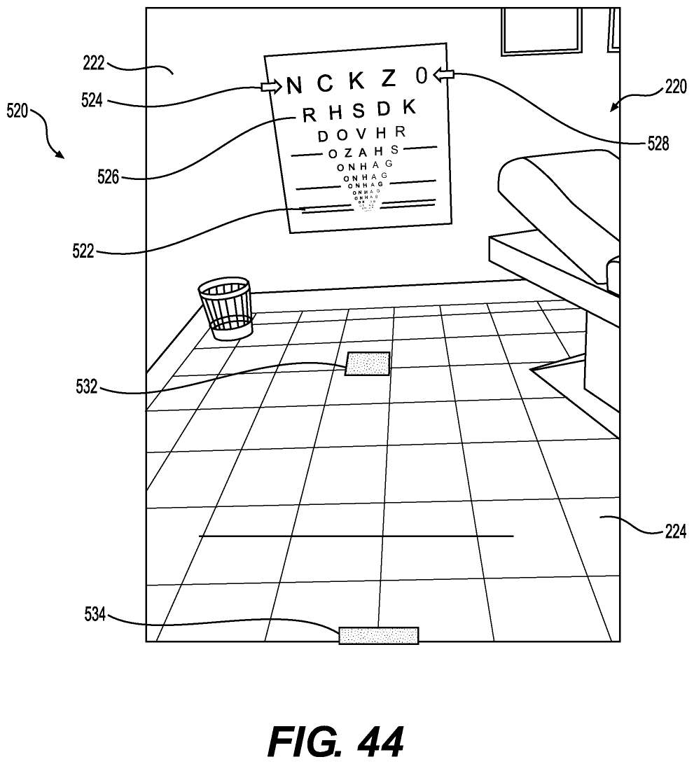

The virtual reality environment 520 of this embodiment is shown in FIG. 44. In this embodiment, there are two positions, a first position 532 and a second position 534, on the virtual floor 224 of the virtual room 220. In this embodiment, the first position 532 and the second position 534 are shown as green squares to indicate the position the user 10 should stand to complete the assessment of this embodiment, but the first position 532 and the second position 534 and other suitable indications may be used including, for example, lines dawn on the virtual floor 224. The first position 532 is spaced a suitable distance from the virtual wall 222 for patients (users 10) with poor vision. In this embodiment, the first position 532 is configured to simulate a distance of 1 meter from the virtual wall 222. The second position 534 is spaced a suitable distance from the virtual wall 222 for other patients (users 10). In this embodiment, the second position 534 is configured to simulate a distance of 4 meters from the virtual wall 222. The user 10 stands at the appropriate position (first position 532 or second position 534) to take the visual acuity assessment.

The visual acuity assessment could be managed by a technician. When managed by a technician, the technician can toggle between different eye charts using a computer (not shown) communicatively coupled to the user system 130. Any suitable connection may be used, including for example, the internet, where the technician is connected to the user system 130 using a web interface operable on a web browser of the computer. The technician can toggle between the plurality of different eye charts (three in this embodiment), and virtual reality system 100, in response to an input received from the user interface associated with the technician, displays one of the plurality of eye charts as the virtual eye chart 522 on the virtual wall 222. The technician can move an arrow 528 up or down to indicate which line the user 10 should read, and virtual reality system 100, in response to an input received from the user interface associated with the technician, positions the arrow 528 to point to a row of the virtual eye chart 522. The arrow 528 is an example of an indication indicating which line of the virtual eye chart 522 the user 10 should read, and this embodiment is not limited to using an arrow 528 as the indication. Where the technician is located locally with the user 10, the technician could use the controller 120 of the virtual reality system 100 to move the arrow 528.

The process for moving the arrow 528 is not so limited and may, for example, be automated. In this embodiment, for example, the virtual reality system 100 may include a microphone and include voice recognition software. The virtual reality system 100 could determine, using the voice recognition software, if the user 10 says the correct letter as the user 10 reads aloud the letters on the virtual eye chart 522. The virtual reality system 100 then moves the arrow 528 starting at the top line and moving down the chart as correct letters are read.

The performance metrics for visual acuity assessment of this embodiment may be measured in the number of characters (such as the number of alphanumeric characters) correctly identified and the size of those characters. As with the low vision visual acuity assessment, the performance metric related to the size of the character may be calculated as MAR and LogMAR, as discussed above.

### Oculomotor Instability Assessment

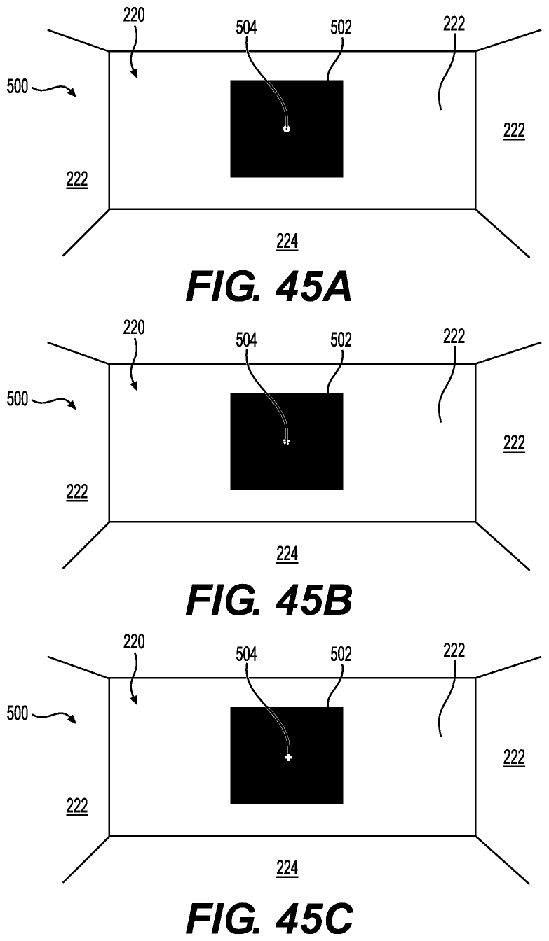

The head mounted display 110 may include the ability to track users eye movements using a sensor 114 of the head mounted display 110 while the user 10 performs tasks. The virtual reality system 100 then generates eye movement data. The eye movement data can be uploaded (automatically, for example) to a server using the virtual reality system 100 and a variety of outcome variables can be calculated that evaluate oculomotor instability. The oculomotor instability assessment of this embodiment may use the virtual reality environment 500 of the low vision visual acuity assessment discussed above. The user 10 stares at a target 504 which may be the virtual screen 502, which is blank, or another object, such as the alphanumeric character 512, for example. The oculomotor instability assessment is not limited to these environments and other suitable targets for the user 10 to stare at may be used. FIGS. 45A, 45B, and 45C, for example, show examples of other targets 504 which may be used in the virtual reality environment 500 of this embodiment. In FIG. 45A the target 504 is a small, red circle located on a black background (virtual screen 502). In FIG. 45B the target 504 is a small, red segmented circle located on a black background (virtual screen 502). In FIG. 45C the target 504 is a small, red cross located on a black background (virtual screen 502).

As the user 10 stares at the target, the head mounted display 110 tracks the location of the center of the pupil and generates eye tracking data. The eye tracking data can then be analyzed to calculate performance metrics. One such performance metric may be median gaze offset, which is the median distance from actual pupil location to normal primary gaze (staring straight ahead at the target). Another performance metric may be variability (2 SD) of the radial distance between actual pupil location and primary gaze. Other metrics could be the interquartile range (IQR) or the median absolute deviation from the normal primary gaze.

### Item Search Assessment

Geographic atrophy, Glaucoma, or any (low vision) ocular condition, including inherited retinal dystrophies, may also be assessed using the virtual reality system 100 discussed herein. One such assessment may include presenting the user 10 with a plurality of scenes (or scenarios) and asking the user 10 to identify a one virtual item of a plurality of virtual items within the scene. In such scenarios, the user 10 could virtually grasp or pick up the item, point at the item and click a button 122 of the controller 120, and/or read or say something that will confirm they saw the item. When the head mounted display 110 is equipped with eye tracking software and devices, the virtual reality system 100 can monitor the eye of the user 10 and, if the user 10 fixated on the intended object, determine that the user 10 saw the requested item. In this embodiment, the virtual reality system 100 and virtual reality environment 550 for this test may include audio prompts to tell the participant what item to identify.

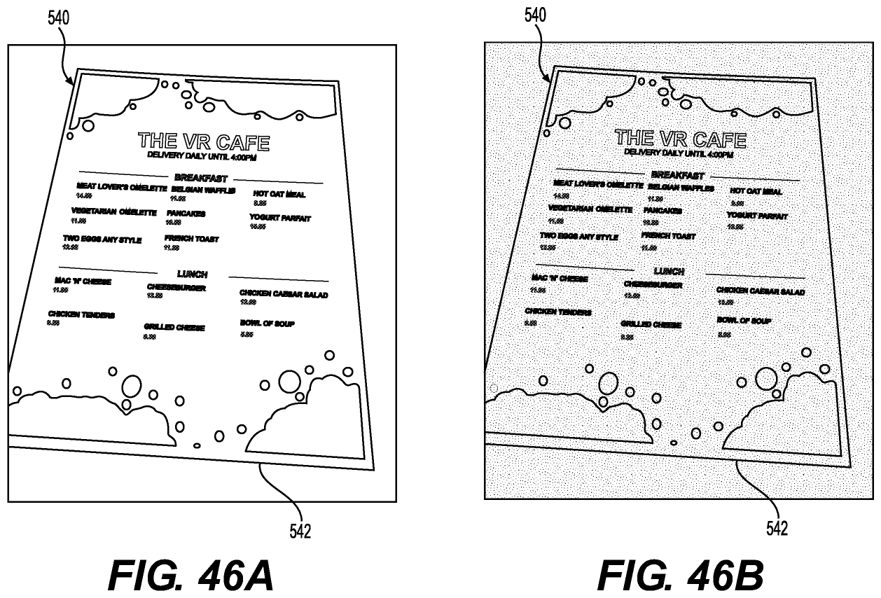

Any suitable scenes or scenarios could be used. As with the virtual navigation course 202 discussed above, each of the scenes of the virtual reality environment 550 could have various different luminance levels to test the user 10 in both well-lit and poorly lit environments. In this embodiment, the luminance level may be chosen in randomized fashion. FIGS. 46A and 46B show an example of a scenario of this embodiment. FIG. 46A is a high (well-lit) luminance level and FIG. 46B is a low (poorly lit) luminance level. In this scenario, a virtual menu 542 is be presented and the user is asked to identify an aspect of the menu. For example, the user 10 may be asked to identify the cost of an item such as the cost of the “Belgian Waffles,” for example. The virtual reality system 100 identifies that the user 10 has identified the item when it receives confirmation that the user has identified $11.95, such as by receiving an audio response from the user 10 or identifying that the user 10 has pointed to the correct entry and pressed a button 122 of the controller 120.

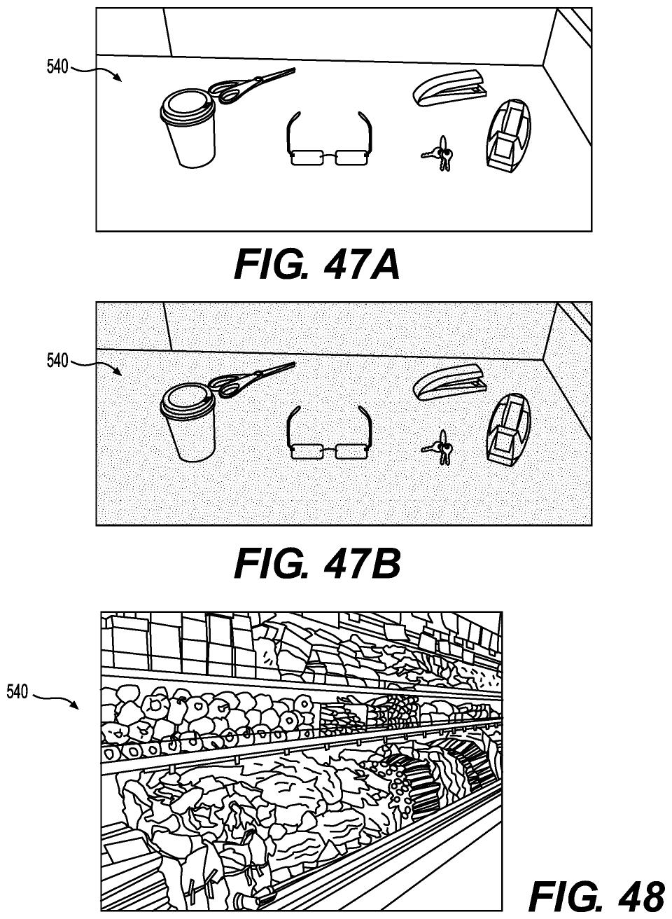

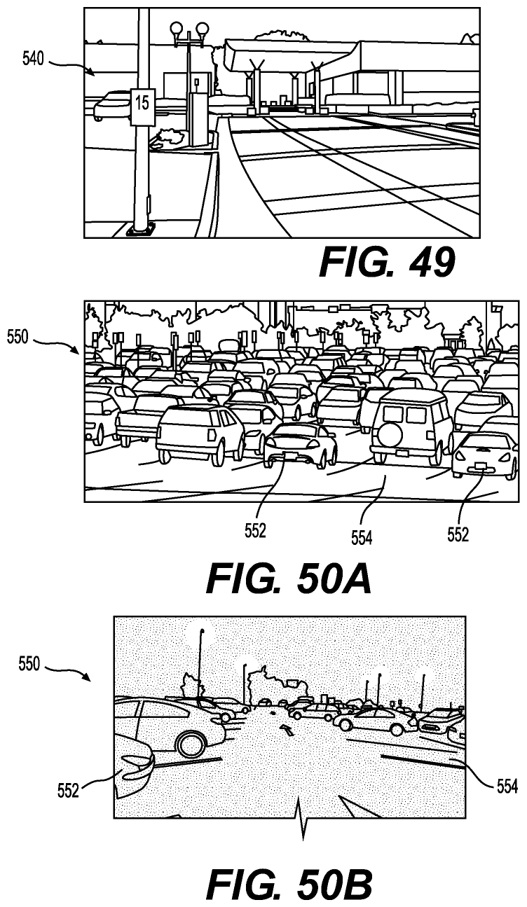

Another scenario includes, for example, a plurality of objects arrayed on a table, such as the objects shown in FIGS. 47A and 47B. FIG. 47A is a high (well-lit) luminance level, and FIG. 47B is a low (poorly lit) luminance level. The user 10 is then asked to identify one of the objects, such as the keys. In still a further scenario, the user 10 may be asked to “grab” or identify an item on a shelf, such as the shelf at a store, for example. FIG. 48 shows a produce cabinet/shelf in a produce isle and the user 10 may be asked to grab a red pepper, for example. Yet another example scenario is shown in FIG. 49 and includes a roadway with street signs. In this embodiment, the user 10 may be asked to identify a street sign, such as the speed limit sign shown in FIG. 49. Still another example scenario includes tracking a person crossing the street. A plurality of people could be included in the scene and the user 10 tracks one of the moving people. In one embodiment, one person is moving, and the rest are stationary. Numerous other example scenarios include finding glasses in a room, simulating a website and asking the user 10 to find specific item on the page, and finding an item on a map.

Further scenarios may include facial recognition tasks. One type of facial recognition task may be an odd-one-out task, where the user 10 identifies the face that is different (odd one) from others presented. The odd-one-out task could help eliminate effects of memory as compared to other memory tasks. In the odd-one-out facial recognition task, four virtual people may be located in a virtual room 220, such as a room that simulates a hallway, and walk toward the user 10. Alternatively, the user 10 could walk towards the four virtual people. Each of the four virtual people would have the same height, hair, clothing, and the like, but one of the four virtual people would have slightly different facial features (“the odd virtual person”). The user 10 would be asked to identify the odd virtual person, by for example, pointing at the odd virtual person and pressing a button 122 of the controller 120.

### Driving Assessment

Another functional vision assessment that may be used to assess, for example, Geographic atrophy, Glaucoma, or other (low vision) ocular conditions, includes a driving assessment. As with the virtual navigation course 202 and virtual reality environment 550 discussed above, the virtual reality environment 550 could have tasks with various different luminance levels to test the user 10 in both well-lit and poorly lit environments. FIGS. 50A and 50B show an example of a scenario of this embodiment. FIG. 50A is a high (well-lit) luminance level simulating a sunny day, and FIG. 50B is a low (poorly lit) luminance level, simulating night scene with street lights. In this driving assessment, the user 10 is asked to drive in a poorly lit residential street or parking lot as shown in FIGS. 50A and 50B and avoid obstacles, such as cars 552. In another variation of the driving assessment of this embodiment the user 10 may be asked to park in a parking space 554. The virtual reality environment 550 of the driving assessment may thus be a virtual driving course for the user to navigate similarly to the virtual navigation course 202 discussed above, but where the virtual obstacles are cars 552 and other obstacles typically found on a roadway or parking lot.

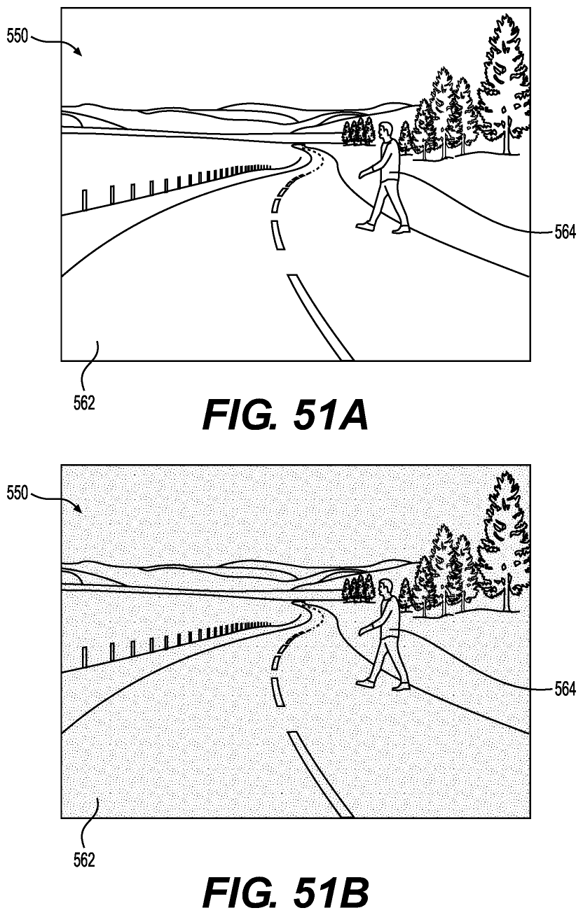

FIGS. 51A and 51B show another example of a scenario of this embodiment. FIG. 51A is a high (well-lit) luminance level simulating a sunny day, and FIG. 51B is a low (poorly lit) luminance level, simulating night scene with street lights. In this scenario, the user 10 is asked to drive down a road 562, such as the gradually curving road 562 shown in FIGS. 51A and 51B. As the user 10 drives (navigates) the road 562, an object appears and starts walking across the road 562. In this embodiment, the object crossing the road 562 is a virtual person 564, but any suitable object may be used, including those that typically cross roads including animals, such as deer. The virtual person 564 would appear after a predetermined amount of time, which may be varied between different instances of the user 10 navigating the virtual road 562. The user 10 then breaks to attempt to avoid a collision with the virtual person 564.

The controller 120 may be used for driving. For example, different buttons 122 of the controller 120 may be used to accelerate and brake and the controller 120 rotated (or the thumb stick 124 used) to steer. As shown in FIG. 1, the virtual reality system 100 of this embodiment, however, may also be equipped with a pedal assembly 150 and steering assembly 160 coupled to the user system 130. Each of the pedal assembly 150 and steering assembly 160 may be coupled to the user system 130 using any suitable means including those discussed above for the controller 120. The pedal assembly 150 includes an accelerator pedal 152 (gas pedal) and a brake pedal 154. The accelerator pedal 152 and the brake pedal 154 are input devices similar to the buttons 122 of the controller 120 and send signals to the user system 130 indicating that the user 10 intends to accelerate or brake, respectively. The pedal assembly 150 may be located on the physical floor of the physical room 20, such as under a table placed in the physical room 20, and operated by the feet of the user 10. The steering assembly 160 of this embodiment includes a steering wheel 162 that is operated by the hands of the user to provide input to the user system 130 that the user 10 intends to turn. The steering wheel 162 of this embodiment is an input device similar to the accelerator pedal 152 and brake pedal 154. The steering assembly 160 may be located on a table placed in the physical room 20 with the user 10 seated next to the table.

The performance metrics used in this embodiment may be based on reaction time. For example, the virtual reality system 100 may measure the reaction time of the user 10 by comparing the time the virtual person 564 starts crossing the road 562 with the time the virtual reality system 100 receives input from the pedal assembly 150 that the user 10 has depressed the brake pedal 154. Other suitable performance metrics may also be used, including for example, whether or not the user 10 successfully brakes in time to prevent a collision with the virtual person 564.

Although this invention has been described with respect to certain specific exemplary embodiments, many additional modifications and variations will be apparent to those skilled in the art in light of this disclosure. It is, therefore, to be understood that this invention may be practiced otherwise than as specifically described. Thus, the exemplary embodiments of the invention should be considered in all respects to be illustrative and not restrictive, and the scope of the invention to be determined by any claims supportable by this application and the equivalents thereof, rather than by the foregoing description.

What is claimed is:

1. A method of evaluating visual impairment of a user comprising:
generating, using a processor, a virtual navigation course for the user to navigate;
displaying portions of the virtual navigation course on a head-mounted display as the user navigates the virtual navigation course, the head-mounted display being communicatively coupled to the processor; and
measuring the progress of the user as user navigates the virtual navigation course using at least one performance metric.

2. The method of claim 1, wherein the performance metric includes at least one of the time for the user to navigate the virtual navigation course and the total distance traveled to navigate the virtual navigation course.

3. The method of claim 1, wherein the virtual navigation course includes a plurality of virtual objects.

4. The method of claim 3, further comprising determining, using the processor, when the user collides with one virtual object of the plurality of virtual objects, as the user navigates the virtual navigation course, based on input received from at least one sensor communicatively coupled with the processor, the at least one performance metric includes the number of collisions with the virtual objects.

5. The method of claim 3, wherein the virtual objects are virtual obstacles, the virtual obstacles being arranged to define a path of the virtual navigation course.

6. The method of claim 5, wherein a plurality of the virtual obstacles is a plurality of virtual furniture.

7. The method of claim 6, wherein the plurality of virtual furniture includes at least one of a chair, a table, a bookcase, a bench, a sofa, and a television.

8. The method of claim 6, wherein the plurality of virtual furniture includes a first piece of furniture having a first simulated height and a second piece of furniture having a second simulated height higher than the first simulated height.

9. The method of claim 8, wherein at least one piece of furniture of the plurality of simulated furniture has a simulated height of at least 5 feet.

10. The method of claim 8, wherein at least one simulated furniture of the plurality of simulated furniture has a simulated height between 18 inches and 36 inches.

11. The method of claim 5, wherein at least one of the virtual obstacles is a removeable virtual obstacle.

12. The method of claim 11, further comprising removing, using the processor, removable virtual obstacle from the virtual navigation course in response to an action taken by the user.

13. The method of claim 12, further comprising determining the position of the head of a user based upon data received from a sensor, wherein the processor removes a simulated obstacle from the virtual navigation course when the sensor transmits to the processor that the user has positioned the removable virtual obstacle within the center of their field of view for a predetermined amount of time.

14. The method of claim 12, further comprising determining the position of the head of a user based upon data received from a sensor, wherein the processor removes a simulated obstacle from the virtual navigation course when the sensor transmits to the processor that the user has positioned the removable virtual obstacle within the center of their field of view and upon receipt of user input from a user input device.

15. The method of claim 14, wherein the user input device is a controller configured to be held in a hand of the user, the controller including a button, and
wherein the processor is configured to receive the user input from the user in response to the user pressing the button of the controller.

16. The method of claim 11, further comprising determining, using the processor, when the user collides with the removable virtual obstacle, as the user navigates the virtual navigation course, based on input received from at least one sensor communicatively coupled with the processor, the at least one performance metric includes the number of collisions with the removable virtual obstacles.

17. The method of claim 11, wherein the removable virtual obstacle is a toy.

18. The method of claim 17, wherein the virtual navigation course further includes a simulated floor, removeable virtual obstacles being located on the simulated floor.

19. The method of claim 1, wherein the virtual navigation course includes a plurality of virtual rooms.

20. The method of claim 19, wherein a first room of the plurality of virtual rooms has a first luminance level and a second room of the plurality of virtual rooms has a second luminance level, the second luminance level being different from the first luminance level.

21. The method of claim 13, wherein a first room of the plurality of virtual rooms has a first contrast level and a second room of the plurality of virtual rooms has a second contrast level, the second contrast level being different from the first contrast level.

22. A non-transitory computer readable storage medium comprising a sequence of instructions for a processor to execute the method of claim 1.

23. A method of evaluating visual impairment of a user comprising:
generating, using a processor, a virtual reality environment including a virtual object having a directionality;
displaying the virtual reality environment including the virtual object on a head-mounted display, the head-mounted display being communicatively coupled to the processor;
increasing, using the processor, the size of the virtual object displayed on the head-mounted display; and
measuring at least one performance metric when the processor receives an input that a user has indicated the directionality of the virtual object.

24. The method of claim 23, wherein the virtual object is an alphanumeric character and increasing the size of the virtual object includes increasing the size of the alphanumeric character.

25. The method of claim 23, wherein the virtual object is a grating having a plurality of bars and increasing the size of the virtual object includes increasing the width of plurality of bars.

26. The method of claim 25, wherein the plurality of bars of the grating are one of horizontal and vertical.

27. The method of claim 23, wherein the processor is communicatively coupled to a sensor and the sensor is configured to detect when the user is pointing in a direction and transmit an input corresponding to the direction user is pointing to the processor.

28. The method of claim 27, wherein the processor is communicatively coupled to a controller having a button and the sensor is configured to detect the direction the user is pointing and transmit the input corresponding to the direction user is pointing to the processor when the button is pressed.

29. A non-transitory computer readable storage medium comprising a sequence of instructions for a processor to execute the method of claim 23.

30. A method of evaluating visual impairment of a user comprising:
generating, using a processor, a virtual reality environment including a virtual eye chart located on a virtual wall, the virtual eye chart having a plurality of lines each of which include at least one alphanumeric character, the at-least-one alphanumeric character in a first line of the eye chart being a different size than the at-least-one alphanumeric character in a second line of the eye chart;
displaying the virtual reality environment including the virtual eye chart and virtual wall on a head-mounted display, the head-mounted display being communicatively coupled to the processor;
displaying, on a head-mounted display, an indication in the virtual reality environment to instruct a user to read one line of the eye chart; and
measuring the progress of the user as user reads the at least one alphanumeric character of the line of the eye chart using at least one performance metric.

31. The method of claim 30, wherein the processor is communicatively coupled to a microphone and measuring the progress of the user by voice recognition.

32. The method of claim 30, wherein the indication indicates that the first line of the eye chart should be read,
the processor is communicatively coupled to a microphone and measuring the progress of the user by voice recognition, and
wherein, in response to the user correctly reading the at least one alphanumeric character of the first line, the indication is moved to indicate that the user should read the second line of the eye chart.

33. The method of claim 30, wherein the virtual reality environment includes a virtual floor and a line on the virtual floor indicating where the user should stand.

34. A non-transitory computer readable storage medium comprising a sequence of instructions for a processor to execute the method of claim 30.

35. A method of evaluating visual impairment of a user comprising:
generating, using a processor, a virtual reality environment including a target;
displaying the virtual reality environment including the target on a head-mounted display, the head-mounted display being communicatively coupled to the processor and including eye-tracking sensors;
tracking the center of the pupil with the eye-tracking sensors to generate eye tracking data as the user stares at the target; and
measuring the visual impairment of the user based on the eye tracking data.

36. A non-transitory computer readable storage medium comprising a sequence of instructions for a processor to execute the method of claim 35.

37. A method of evaluating visual impairment of a user comprising:
generating, using a processor, a virtual reality environment including a virtual scene having a plurality of virtual objects arranged therein;
displaying the virtual reality environment including the virtual scene and the plurality of virtual objects on a head-mounted display, the head-mounted display being communicatively coupled to the processor; and
measuring the performance of the user using at least one performance metric when the processor receives an input that a user has selected an object of the plurality of virtual objects.

38. The method of claim 37, further comprising instructing the user which virtual object to select.

39. The method of claim 38, wherein the performance metric includes whether the user selected the virtual object instructed to be selected.

40. A non-transitory computer readable storage medium comprising a sequence of instructions for a processor to execute the method of claim 37.

41. A method of evaluating visual impairment of a user comprising:
generating, using a processor, a virtual driving course for the user to navigate;
displaying portions of the virtual driving course on a head-mounted display as the user navigates the virtual navigation course, the head-mounted display being communicatively coupled to the processor; and
measuring the progress of the user as user navigates the virtual navigation course using at least one performance metric.

42. A non-transitory computer readable storage medium comprising a sequence of instructions for a processor to execute the method of claim 31.
